SEQUENCE LISTING (14 sequences)

SEQ ID NO: 1 - DNA, 22 bp, Artificial Sequence
  synthetic primer
gcaactgttcctgaactcaact

SEQ ID NO: 2 - DNA, 21 bp, Artificial Sequence
  synthetic primer
atcttttggggtccgtcaact

SEQ ID NO: 3 - DNA, 20 bp, Artificial Sequence
  synthetic primer
tccagttgccttcttgggac

SEQ ID NO: 4 - DNA, 20 bp, Artificial Sequence
  synthetic primer
gtactccagaagaccagagg

SEQ ID NO: 5 - DNA, 21 bp, Artificial Sequence
  synthetic primer
gctcttactgactggcatgag

SEQ ID NO: 6 - DNA, 20 bp, Artificial Sequence
  synthetic primer
cgcagctctaggagctagtg

SEQ ID NO: 7 - DNA, 22 bp, Artificial Sequence
  synthetic primer
cacactcagatcatcttctcaa

SEQ ID NO: 8 - DNA, 22 bp, Artificial Sequence
  synthetic primer
agtagacaaggtacaacccatc

SEQ ID NO: 9 - DNA, 19 bp, Artificial Sequence
  synthetic primer
tcgtcatcatcgtcgtcca

SEQ ID NO: 10 - DNA, 21 bp, Artificial Sequence
  synthetic primer
agaatgctgtgtcctctgaag

SEQ ID NO: 11 - DNA, 22 bp, Artificial Sequence
  synthetic primer
gactcacacctctcacatcttg

SEQ ID NO: 12 - DNA, 19 bp, Artificial Sequence
  synthetic primer
gtgcccttcagattgttgc

SEQ ID NO: 13 - DNA, 20 bp, Artificial Sequence
  synthetic primer
agatgtggatcagcaagcag

SEQ ID NO: 14 - DNA, 20 bp, Artificial Sequence
  synthetic primer
gcgcaagttaggttttgtca

(19) United States
(12) Patent Application Publication
(10) Pub. No.: US 2021/0244851 A1
(43) Pub. Date: Aug. 12, 2021

(54) COMPOSITIONS AND METHODS FOR TARGETING ANGIOGENESIS FOR FRACTURE NONUNION TREATMENT UNDER INFLAMMATORY DISEASES

(71) Applicants: Jie Shen, St. Louis, MO (US); Jianjun Guan, St. Louis, MO (US)

(72) Inventors: Jie Shen, St. Louis, MO (US); Jianjun Guan, St. Louis, MO (US)

(73) Assignee: Washington University, St. Louis, MO (US)

(21) Appl. No.: 17/170,310

(22) Filed: Feb. 8, 2021

Related U.S. Application Data
(60) Provisional application No. 62/971,820, filed on Feb. 7, 2020.

(51) Int. Cl. A61L 27/22; A61L 27/58; A61L 27/18

(52) U.S. Cl. CPC A61L 27/227; C08L 67/04; A61L 27/18; A61L 27/58

(57) ABSTRACT

Methods and formulations for promoting the healing of fracture nonunion sites under inflammatory conditions. The formulation includes a flexible biodegradable scaffold loaded with therapeutically effective amounts of CXCL12 and SPPI encapsulated in biodegradable microspheres. The method includes applying the formulation directly to a region of a fracture nonunion to effect sustained local release of the therapeutically effective amounts of CXCL12 and SPPI to the fracture nonunion region.

### CROSS-REFERENCE TO RELATED APPLICATIONS

This application claims priority from U.S. Provisional Application Ser. 62/971,820 filed on Feb. 7, 2020, which is incorporated herein by reference in its entirety.

### STATEMENT REGARDING FEDERALLY SPONSORED RESEARCH OR DEVELOPMENT

This invention was made with government support under R01AR075860 awarded by the National Institutes of Health. The government has certain rights in the invention.

### MATERIAL INCORPORATED-BY-REFERENCE

The Sequence Listing, which is a part of the present disclosure, includes a computer-readable form comprising nucleotide and/or amino acid sequences of the present invention. The subject matter of the Sequence Listing is incorporated herein by reference in its entirety.

### FIELD OF THE DISCLOSURE

The present disclosure generally relates to methods and compositions for healing fracture nonunions.

### BACKGROUND

Approximately ten percent of the 16 million fractures occurring annually in the United States do not progress to timely union and encounter prolonged healing or nonunion each year. There are two distinct types of fracture nonunion, atrophic and hypertrophic nonunion, characterized by different radiographical observation and biological properties. Development of hypertrophic nonunion is primarily due to inadequate mechanical stability, leading to excessive fracture callus formation, therefore appropriate mobilization and fixation at the fracture site are usually used in clinics to achieve successful outcomes in hypertrophic nonunion patients. In contrast, atrophic nonunion is characterized by limited fracture callus with poor tissue revascularization, likely secondary to perturbation of normal biological cues. Treatment of atrophic nonunion usually involves complex clinical interventions in practice and often requires multiple surgeries. Thus, atrophic non-union results in significant patient disability and increased cost to the health care system. While bone graft surgeries, mechanical stimulation devices, and therapies using growth factors and stem cells have been developed, atrophic nonunion remains a major clinical challenge for orthopedic surgeons.

A major population affected by atrophic nonunion are patients with inflammatory conditions, e.g., elderly patients, smokers, and diabetic or rheumatoid arthritis (RA) patients. In these patients, the fracture risk is increased due to poor bone quality, highlighting the potential deleterious role of chronic systemic inflammation in fracture repair. This can be observed in pre-clinical models. Indeed, TNFα transgenic mice had impaired bone quality, including reduced cortical thickness which leads to decreased fracture toughness. Additionally, experiments using the mouse cortical defect model showed that bone regeneration was also significantly reduced in RA mice. Studies from patients and rodents have extensively documented that chronic systemic inflammation activates the canonical NF-κB pathway resulting in elevated expression of IL-β, TNFα, and other cytokines which impairs the fracture repair process at least partially through negatively affecting angiogenesis. Although pharmacological anti-cytokine therapies have been developed and are highly effective in RA patients, the impact of these agents on fracture healing in patients with inflammatory arthritis is not known. Pre-clinical animal studies showed a positive effect of the TNFα inhibitor Infliximab on the restoration of callus formation and biomechanical properties of fractured bone in wild-type rats under chronic inflammatory conditions. In contrast, a human cohort study from ankylosing spondylitis patients demonstrated a negative long-term effect of TNFα inhibitor treatment on fracture healing. Therefore, there is an urgent need to develop molecular-based therapies for fracture nonunion, especially for older patients with chronic inflammatory diseases.

### SUMMARY OF THE DISCLOSURE

In one aspect, a formulation for treating a fracture non-union in a patient in need is disclosed that includes a biodegradable scaffold, a therapeutically effective amount of CXCL12, and a therapeutically effective amount of SPPI. The biodegradable scaffold is configured to be applied over a region of the fracture non-union, within a gap of the fracture non-union, and any combination thereof. The biodegradable scaffold is configured for extended-release of the therapeutically effective amounts of CXCL12 and SPPI. In some aspects, the biodegradable scaffold includes a plurality of biodegradable polymer fibers that include a scaffold polymer selected from PCL, PTO, and PLGA. In some aspects, the scaffold polymer is PCL. In some aspects, the biodegradable scaffold has a thickness of about 100 μm. In some aspects, the biodegradable scaffold has a width of about 2 mm. In some aspects, the biodegradable scaffold has a tensile strength of about 29 MPa and Young's modulus of about 111.5 MPa. In some aspects, the formulation further includes a plurality of biodegradable microspheres encapsulating the therapeutically effective amounts of CXCL12 and SPPI. In some aspects, the plurality of biodegradable microspheres has a diameter of about 5 μm. In some aspects, the plurality of biodegradable microspheres includes a microsphere polymer selected from PCL, PTO, and PLGA. In some aspects, the microsphere polymer is PLGA. In some aspects, the plurality of biodegradable microspheres is uniformly distributed throughout the biodegradable scaffold. In some aspects, each biodegradable microsphere encapsulates a mixture comprising CXCL12 and SPPI. In some aspects, the plurality of biodegradable microspheres includes a first portion of microspheres encapsulating CXCL12 and a second portion of microspheres encapsulating SPPI. In some aspects, the biodegradable scaffold is configured for sustained release of the therapeutically effective amounts of CXCL12 and SPPI over a period of about 4 weeks.

In another aspect, a method for treating a fracture non-union in a patient in need is disclosed that includes providing a formulation that includes a biodegradable scaffold, a therapeutically effective amount of CXCL12, and a therapeutically effective amount of SPPI. The scaffold is configured for extended-release of the therapeutically effective amounts of CXCL12 and SPPI. The method further includes applying the biodegradable scaffold over a region of the fracture non-union, within a gap of the fracture non-union, and any combination thereof. In some aspects, the biodegradable scaffold includes a plurality of biodegradable polymer fibers that include PCL scaffold polymer. In some aspects, the biodegradable scaffold has a thickness of about 100 μm and a width of about 2 mm. In some aspects, the formulation further comprises a plurality of biodegradable microspheres uniformly distributed throughout the biodegradable scaffold, in which the plurality of biodegradable microspheres include PLGA encapsulating the therapeutically effective amounts of CXCL12 and SPPI. In some aspects, the biodegradable scaffold is configured for sustained release of the therapeutically effective amounts of CXCL12 and SPPI over a period of about 4 weeks. In some aspects, the method further includes modulating the therapeutically effective amounts of CXCL12 and SPPI by varying a number of layers of the biodegradable scaffold applied over the region of the fracture non-union, within a gap of the fracture non-union, and any combination thereof.

Other objects and features will be in part apparent and in part pointed out hereinafter.

### DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A contains representative microscope images of fracture callus sections of control and RA mice at 10 and 14 dpf (n=5) with ABH/OG staining. Scale bar, 200 μm.

FIG. 1B contains graphs summarizing histomorphometric quantification of cartilage and bone area performed on the microscopic images of FIG. 1A at 10 dpf from the control and RA mice (n=5). The results were normalized to the controls. *p<0.05 compared with control by student's t-test.

FIG. 1C is a graph summarizing histomorphometric quantification of bone area performed on the microscopic images of FIG. 1A at 14 dpf from the control and RA mice (n=5). The results were normalized to the controls. *p<0.05 compared with control by student's t-test.

FIG. 1D are images of a MicroCT assessment of mineralized bone within fracture callus at 21 dpf from the control and RA mice (n=5).

FIG. 1E contains graphs summarizing the quantification of bony callus volume and relative BV/TV ratio based on the microCT assessment of FIG. 1D. The results were normalized to the controls. *p<0.05 compared with control by student's t-test.

FIG. 1F contains representative microscope images of immunohistochemical staining for COL3A1 on 21 dpf fracture callus from the control and RA mice. Scale bar, 200 μm.

FIG. 1G contains graphs summarizing the max torque and displacement at max torque obtained from biomechanical torsion testing of the control and RA fractures at 28 dpf (n=5). The results were normalized to the controls. *p<0.05 compared with control by student's t-test. Scale bar, 200 μm.

FIG. 2A contains images of a MicroCT assessment of newly formed vessels within 10 dpf fracture callus from the control and RA mice.

FIG. 2B contains graphs summarizing the quantification of vessel counts in 10 dpf control and RA fracture callus (n=5) based on the microCT assessment of FIG. 2A. The result was normalized to the controls. *p<0.05 compared with control by student's t-test.

FIG. 2C contains microscopic images of 10 dpf fracture callus from the control and RA mice with immunohistochemical staining for Endomucin. Scale bar, 200 μm.

FIG. 2B contains graphs summarizing the quantification of vessel counts in 10 dpf control and RA fracture callus (n=5) based on the microCT assessment of FIG. 2A. The result was normalized to the controls. *p<0.05 compared with control by student's t-test.

FIG. 2D contains graphs summarizing the quantification of vessel counts in 10 dpf control and RA fracture callus (n=5) based on an assessment of the immunohistochemical images of FIG. 2C. The result was normalized to the controls. *p<0.05 compared with control by student's t-test.

FIG. 2E contains representative images of angiogenesis proteasome array from cultured medium of chondrocytes following with vehicle and IL-1β treatment.

FIG. 2F contains a graph summarizing the quantification of blot intensity from the angiogenesis proteasome arrays (n=4) of FIG. 2E. All results were normalized to the controls. *p<0.05 compared with control by student's t-test. Scale bar, 200 μm.

FIG. 3A contains graphs summarizing the relative expression of Spp1 and CXCL12 in 10 dpf fracture callus from the control and RA mice (n=4) based on real-time qPCR analyses. The mRNA levels were normalized to that of Actb and then were normalized to the control group. *p<0.05 compared with control by student's t-test.

FIG. 3B contains representative microscope images with immunohistochemical staining for SPP1 and CXCL12 on 10 dpf fracture callus from control and RA mice.

FIG. 3C contains representative images of HUVEC migration and tube formation assays using culture medium from vehicle and IL-1β treated chondrocytes, supplemented with SPP1, CXCL12, and SPP1+CXCL12. All results were normalized to the controls. *p<0.05 compared with control by two-way ANOVA. Scale bar, 200 μm.

FIG. 3D is a graph summarizing the quantification of HUVEC migration based on the images of FIG. 3C.

FIG. 3E contains graphs summarizing tube number and tube length (n=3) based on the images of FIG. 3C. All results were normalized to the controls. *p <0.05 compared with control by two-way ANOVA. Scale bar, 200 μm.

FIG. 4A is a schematic illustration of an apparatus used for PCL scaffold fabrication that includes simultaneous electrospinning and electrospraying systems.

FIG. 4B contains representative confocal images of PLGA microspheres encapsulated with growth factors; PLGA: red; growth factor: green. Scale bar, 200 μm.

FIG. 4C contains representative SEM images of the PCL scaffold.

FIG. 4D contains graphs summarizing release profiles of SPP1 and CXCL12 from PCL scaffolds loaded with SPP1 and CXCL12 (n=5).

FIG. 5A is a schematic illustration of a collagen construct used to create a 3D cell culture environment for examining the impact of released growth factors on HUVEC cell migration and tube formation.

FIG. 5B contains graphs summarizing the quantification of HUVEC cell numbers at different depths in the collagen gel (n=5) shown illustrated in FIG. 5A. All results were normalized to the controls. *p<0.05 compared with control by two-way ANOVA.

FIG. 5C contains representative images of HUVEC lumen formation in collagen gels.

FIG. 5D contains graphs summarizing the quantification of lumen density at different depths in the collagen gel (n=5). *p<0.05 compared with control by two-way ANOVA. Scale bar, 200 μm.

FIG. 6A contains an image of a PCL scaffold with or without SPP1 and CXCL12 before (left) and after (right, arrow) application to a fractured bone in an RA mouse.

FIG. 6B contains MicroCT images of newly formed vessels within a 10 dpf fracture callus from the RA mice treated with scaffold with or without SPP1 and CXCL12 (n=5).

FIG. 6C contains graphs summarizing the quantification of vessel counts in 10 dpf RA fracture callus (n=5) based on the microCT assessment of FIG. 6B. Results were normalized to the scaffold only group. *p<0.05 compared with control by student's t-test.

FIG. 6D contains microscopic images of 10 dpf fracture callus from the RA mice with immunohistochemical staining for Endomucin.

FIG. 6E contains graphs summarizing the quantification of vessel counts in 10 dpf RA fracture callus (n=5) based on the immunohistochemical assessment of FIG. 6D. Results were normalized to the scaffold only group. *p <0.05 compared with control by student's t-test.

FIG. 6F contains microscopic images of 10 dpf fracture callus with ABH/OG staining of fracture callus sections from RA mice scaffold-treated with or without SPP1 and CXCL1 (n=5). Scale bar, 200 μm.

FIG. 6G contains a graph summarizing the histomorphometric quantification of bone area performed on the images of FIG. 6F. The results were normalized to the scaffold-only (control) group.

FIG. 6H contains graphs summarizing max torque and displacement at max torque measured during biomechanical torsion testing of the RA fractures treated with scaffold with or without SPP1 and CXCL12 at 28 dpf (n=8). All results were normalized to the scaffold-only (control) group. *p<0.05 compared with control by student's t-test.

FIG. 7A contains images of Relb-Luc reporter mice were used to visualize the NF-κB activity in vivo following PBS (Ctrl) and K/B×N (RA) administration.

FIG. 7B is a graph summarizing the quantification of luciferase intensity in the lower limb of Relb-Luc reporter mice at the indicated time points (n=3) based on analysis of the images of FIG. 7A. *p<0.05 compared with control by student's t-test.

FIG. 8 contains graphs summarizing the results of real-time qPCR analyses performed to determine the relative expression of ll1b, ll6, ll10, and Tnfa in 7 dpf fracture callus from control and RA mice (n=4). mRNA levels were normalized to that of Actb (internal control) and then were normalized to the control group. *p<0.05 compared with control by student's t-test.

FIG. 9 contains a series of MicroCT images of mineralized bone within fracture callus at 10 and 14 dpf from control and RA mice (n=5).

FIG. 10A contains representative images of angiogenesis proteasome arrays obtained using cultured medium of osteoblasts following vehicle and IL-1β treatment.

FIG. 10B is a graph summarizing the quantification of blot intensity from angiogenesis proteasome array (n=4) of FIG. 10A. All results were normalized to the controls. *p<0.05 compared with control by student's t-test.

FIG. 11A contains western blot images showing the protein levels of SPP1 and CXCL12 in primary chondrocytes treated with IL-6 for 48 hours.

FIG. 11B contains western blot images showing the protein levels of SPP1 and CXCL12 in primary chondrocytes treated with TNFα for 48 hours.

FIG. 12 contains graphs summarizing the quantification of cell proliferation and apoptosis from HUVEC cells under the culture of medium collected from vehicle and IL-1β treated chondrocytes. All results were normalized to the controls.

FIG. 13 is a graph summarizing tensile stress and strain of PCL scaffolds obtained during mechanical testing of PCL scaffold samples.

FIG. 14A contains MicroCT images of newly formed vessels within 10 dpf fracture callus from RA mice treated with scaffolds loaded with SPP1 or CXCL12 (n=5).

FIG. 14B is a graph summarizing the quantification of vessel counts in 10 dpf RA fracture callus (n=5) based on the microCT images of FIG. 14A. Results were normalized to the scaffold only group. *p<0.05 compared with control by two-way ANOVA.

FIG. 14C contains representative microscopic images of fracture callus sections with ABH/OG staining of RA mice treated with scaffolds loaded with SPP1 or CXCL12 at 10 dpf (n=5).

FIG. 14D is a graph summarizing the histomorphometric quantification of bone area performed on the 10 dpf fracture callus sections from the RA mice treated with scaffolds loaded with SPP1 or CXCL12 (n=5) shown in FIG. 14C. Results were normalized to the scaffold group. *p<0.05 compared with control by two-way ANOVA. Scale bar, 200 μm.

Those of skill in the art will understand that the drawings, described below, are for illustrative purposes only. The drawings are not intended to limit the scope of the present teachings in any way.

### DETAILED DESCRIPTION

The disclosed method enables the promotion of healing fracture nonunion sites under inflammatory conditions by administering CXCL12 and SPP1 within sustained-release bioscaffolds to the fracture nonunion sites to facilitate bony callus formation and bone union.

Our findings highlight local delivery of SPP1 and CXCL12 as an important therapeutic option to improve the angiogenesis and treat fracture atrophic nonunion, especially under inflammatory conditions

During normal fracture repair, chondrocytes and osteoblasts are the primary cells that secrete angiogenic factors, recruiting endothelial cells and facilitating angiogenesis and vasculogenesis. As a key initial step, a re-established vascular network brings oxygen and nutrients to facilitate bone regeneration as well as osteoprogenitors, osteoblasts, and other cells necessary for callus maturation and bone formation. However, under inflammatory conditions, insufficient re-vascularization occurs, leading to atrophic nonunion. Through an unbiased in vitro screen of angiogenic factors, described in the Examples below, secreted phosphoprotein 1 (SPP1) and C-X-C motif chemokine ligand 12 (CXCL12) were found to be the two factors most down-regulated by IL-1β treatment, suggesting SPP1 and CXCL12 as potential targets of inflammation in chondrocytes. SPP1 and CXCL12 are both highly expressed in chondrocytes and osteoblasts during fracture repair and recent rodent studies suggest that SPP1 and CXCL12 contribute to fracture healing through improvement of neovascularization.

In order to target the angiogenesis process, biodegradable scaffolds capable of continuously releasing SPP1 and CXCL12 locally at the fracture site were developed. As described in the Examples below, sustained delivery of SPP1 and CXCL12 accelerated fracture union in an RA mouse model and restored the biomechanical properties at the fracture site, therefore highlighting this approach as a potential therapeutic strategy to treat atrophic fracture nonunion in patients with inflammatory diseases.

SPP1 and CXCL12 have been demonstrated as chemokines to stimulate angiogenesis both during normal organ development and under pathological conditions, such as various cancers. SPP1 itself can recruit endothelial cells to form new blood vessels while it can also attract macrophages and function synergistically with other cytokines derived from macrophages to promote angiogenesis. Similarly, CXCL12 recruits CXCR4 positive endothelial cells and facilitates angiogenesis. In regard to fracture healing, it has also been shown that global knockout of either Spp1 or Cxcl12 alone led to angiogenesis defect and impaired fracture healing in mice, implicating the positive role of SPP1 and CXCL12 on angiogenesis and fracture healing. There is increasing evidence that SPP1 and CXCL12 expression is upregulated in various tissues under inflammatory conditions. Particularly in the RA patients, high concentrations of SPP1 and CXCL12 were detected in synovial fluid and both SPP1 and CXCL12 were found overexpressed in RA synovial cells, which in turn leads to excessive blood vessel invasion and synovial joint destruction. However, in contrast to the responses to inflammatory stimuli from synovial cells, SPP1 and CXCL12 expression was specifically reduced in chondrocytes by IL-1β. In accordance with these in vitro observations, the expression of SPP1 and CXCL12 was also found to decrease in the RA callus. These findings highlight supplementation of SPP1 and CXCL12 as a promising therapeutic approach to treat atrophic nonunion, particularly chronic inflammation-induced nonunion.

The controlled release of SPP1 and CXCL12 locally via the disclosed PCL scaffold restores angiogenesis and fracture repair in RA mice, as described in the Examples below. In various aspects, the controlled release scaffold provides a potential therapeutic approach to treat impaired angiogenesis and fracture nonunion in patients, especially under inflammatory conditions.

Formulation

In various aspects, a formulation is disclosed for treating a fracture non-union in a patient in need, in particular in patients with inflammatory diseases including, but not limited to, elderly patients, smokers, and diabetic or rheumatoid arthritis (RA) patients. The formulation includes a biodegradable scaffold loaded with a therapeutically effective amount of at least one of CXCL12 and SPPI. In some aspects, the biodegradable scaffold may be loaded with additional active compounds to facilitate the treatment of the fracture non-union including, but not limited to, heparin. In various aspects, the formulation is configured to be applied over a region of the fracture non-union, within a gap of the fracture non-union, or both.

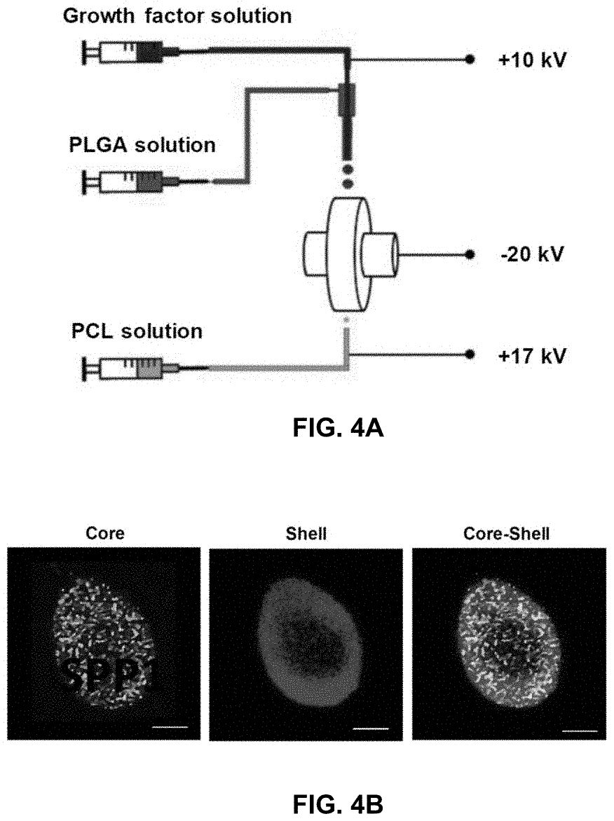

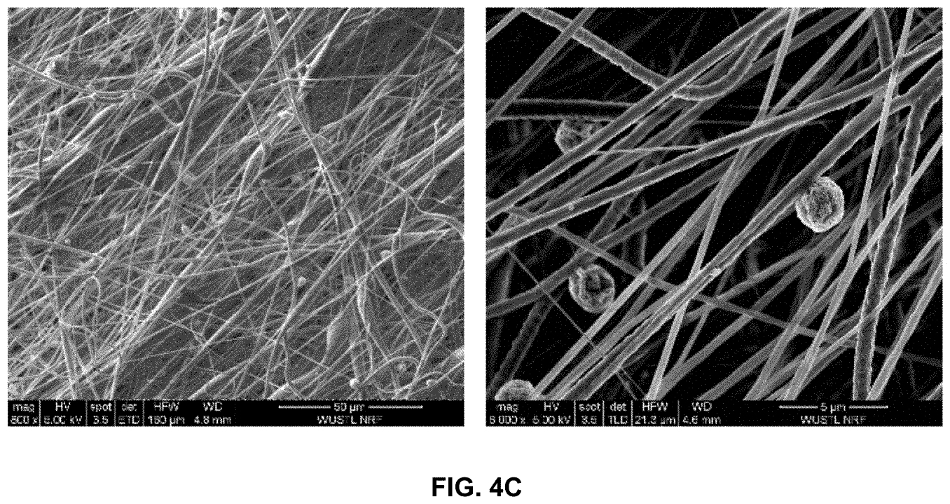

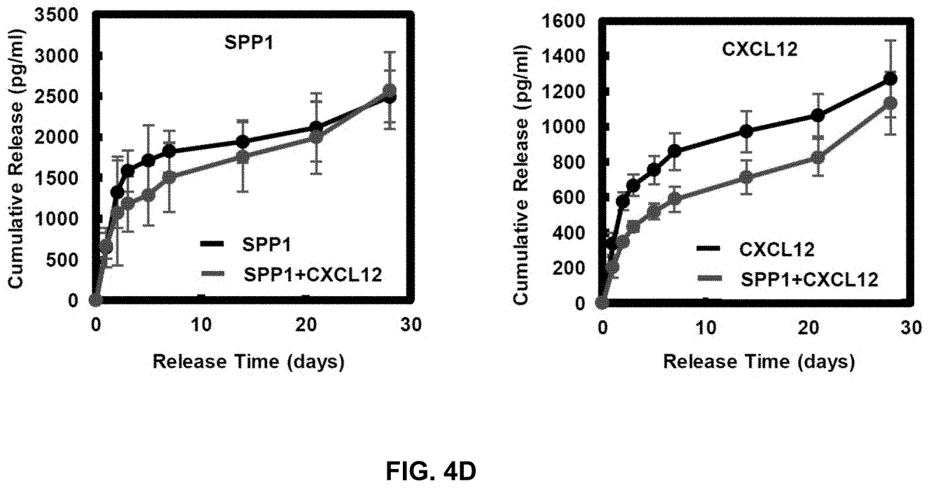

In various aspects, the biodegradable scaffold contains a plurality of flexible biodegradable polymer fibers formed using one or more biodegradable polymers, as illustrated in FIG. 4C. Any suitable flexible biodegradable polymer may be used to form the biodegradable scaffold without limitation including, but not limited to, polycaprolactone (PCL), polyterephthaloyl oxalamidrazone (PTO), and poly(lactic-co-glycolic acid) (PLGA). In one exemplary aspect, the biodegradable scaffold is formed using PCL.

In various aspects, the functional properties of the biodegradable scaffold may be modulated by varying at least one characteristic of the flexible biodegradable fibers within the scaffold including, but not limited to, selection of biodegradable polymer used to form the fibers, fiber diameter, and fiber density. Non-limiting examples of functional properties of the biodegradable scaffold include mechanical properties, sustained release profile of the active compounds loaded into the biodegradable scaffold, and resorption time.

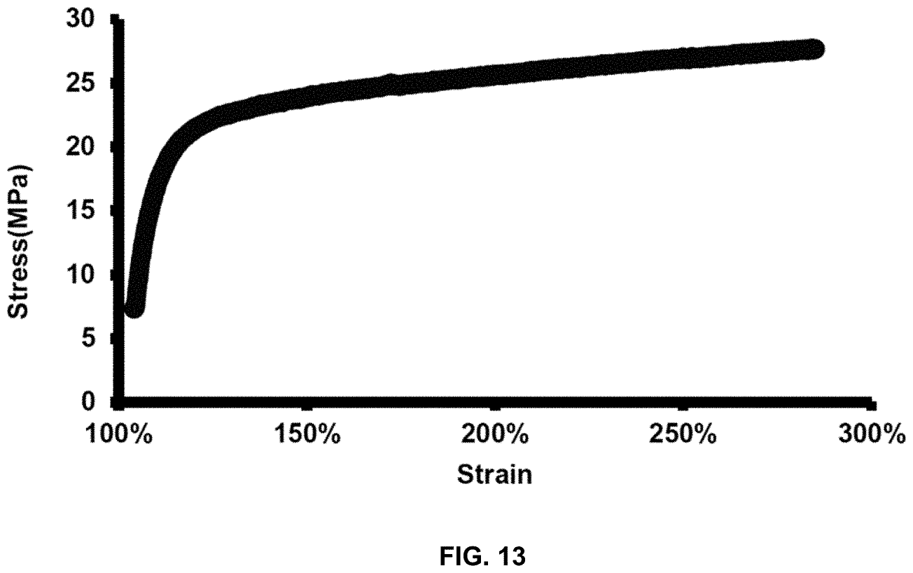

In some aspects, the mechanical properties of the biodegradable scaffold may be selected to essentially match the mechanical properties of periosteum tissue. In one aspect, the biodegradable scaffold has a tensile strength of about 29 MPa and Young's modulus of about 111.5 MPa, as illustrated in FIG. 13. In various other aspects, the biodegradable scaffold has a tensile strength ranging from about 20 MPa to about 40 MPa, from about 22.5 MPa to about 37.5 MPa, from about 25 MPa to about 35 MPa, from about 27.5 MPa to about 32.5 MPa, and from about 28 MPa to about 32 MPa. In various other aspects, the biodegradable scaffold has a tensile strength ranging from about 20 MPa to about 22 MPa, from about 21 MPa to about 23 MPa, from about 22 MPa to about 24 MPa, from about 23 MPa to about 25 MPa, from about 24 MPa to about 26 MPa, from about 25 MPa to about 27 MPa, from about 26 MPa to about 28 MPa, from about 27 MPa to about 29 MPa, from about 28 MPa to about 30 MPa, from about 29 MPa to about 31 MPa, from about 30 MPa to about 32 MPa, from about 31 MPa to about 33 MPa, from about 32 MPa to about 34 MPa, from about 33 MPa to about 35 MPa, from about 34 MPa to about 36 MPa, from about 35 MPa to about 37 MPa, from about 36 MPa to about 38 MPa, from about 37 MPa to about 39 MPa, and from about 38 MPa to about 40 MPa. In various other aspects, the biodegradable scaffold has Young's modulus ranging from about 100 MPa to about 120 MPa, from about 102.5 MPa to about 117.5 MPa, from about 105 MPa to about 115 MPa, from about 107.5 MPa to about 112.5 MPa, and from about 110 MPa to about 112 MPa. In various other aspects, the biodegradable scaffold has a tensile strength ranging from about 100 MPa to about 102 MPa, from about 101 MPa to about 103 MPa, from about 102 MPa to about 104 MPa, from about 103 MPa to about 105 MPa, from about 104 MPa to about 106 MPa, from about 105 MPa to about 107 MPa, from about 106 MPa to about 108 MPa, from about 107 MPa to about 109 MPa, from about 108 MPa to about 110 MPa, from about 109 MPa to about 111 MPa, from about 110 MPa to about 112 MPa, from about 111 MPa to about 113 MPa, from about 112 MPa to about 114 MPa, from about 113 MPa to about 115 MPa, from about 114 MPa to about 116 MPa, from about 115 MPa to about 117 MPa, from about 116 MPa to about 118 MPa, from about 117 MPa to about 119 MPa, and from about 118 MPa to about 120 MPa.

In some aspects, the biodegradable scaffold is provided as a strip with a thickness of about 100 μm and a width of about 2 mm. In various other aspects, the thickness and/or width of the biodegradable scaffold may vary to provide for a modified release profile of active compounds loaded into the scaffold, to provide sufficient size to cover the region of the fracture non-union or any other suitable use. In some aspects, the thickness of the scaffold strip may be at least 50 μm, at least 75 μm, at least 100 μm, at least 125 μm, at least 150 μm, at least 175 μm, or at least 200 μm or more. In other aspects, the thickness of the scaffold strip ranges from about 50 μm to about 70 μm, from about 60 μm to about 80 μm, from about 70 μm to about 90 μm, from about 75 μm to about 85 μm, from about 80 μm to about 90 μm, from about 85 μm to about 95 μm, from about 90 μm to about 100 μm, from about 95 μm to about 105 μm, from about 100 μm to about 110 μm, from about 115 μm to about 120 μm, from about 120 μm to about 130 μm, from about 120 μm to about 140 μm, from about 150 μm to about 170 μm, from about 160 μm to about 180 μm, from about 170 μm to about 190 μm, and from about 180 μm to about 200 μm.

In some aspects, the width of the scaffold strip may be at least 0.5 mm, at least 1 mm, at least 1.5 mm, at least 2 mm, at least 2.5 mm, at least 5 mm, or at least 10 mm or more. In other aspects, the thickness of the scaffold strip ranges from about 0.5 mm to about 1 mm, from about 0.75 mm to about 1.25 mm, from about 1 mm to about 1.5 mm, from about 1.25 mm to about 1.75 mm, from about 1.5 mm to about 2 mm, from about 2 mm to about 4 mm, from about 3 mm to about 5 mm, from about 4 mm to about 6 mm, from about 5 mm to about 7 mm, from about 6 mm to about 8 mm, from about 7 mm to about 9 mm, and from about 8 mm to about 10 mm.

In various aspects, the active compounds may be loaded into the biodegradable scaffold in any suitable form including, but not limited to, free form, complexed with biodegradable carrier moieties, and encapsulated in biodegradable microspheres. In one exemplary aspect, the active compounds are encapsulated with biodegradable polymer microspheres and loaded into the biodegradable scaffold in the encapsulated form. In some aspects, the biodegradable polymer microspheres are uniformly distributed throughout the biodegradable scaffold.

In various aspects, at least one characteristic of the biodegradable microspheres encapsulating the active compounds may be modulated to produce a desired sustained release profile of the active compounds from the biodegradable scaffold. Non-limiting examples of suitable characteristics of the biodegradable microspheres encapsulating the active compounds that may be modulated include the composition of the outer microsphere shell, microsphere diameter, the density of the microspheres within the scaffold, the composition of active compounds and optional excipients encapsulated within the microspheres, and any other suitable characteristic.

In various aspects, the biodegradable microspheres are formed from at least one flexible biodegradable polymer. Any suitable biodegradable polymer may be used to form the biodegradable microspheres without limitation including, but not limited to, polycaprolactone (PCL), polyterephthaloyl oxalamidrazone (PTO), and poly(lactic-co-glycolic acid) (PLGA). In one exemplary aspect, the biodegradable microspheres are formed using PLGA.

In various aspects, the diameter of the biodegradable microspheres is about 5 μm. In other aspects, the diameter of the biodegradable microspheres may range from about 1 μm to about 3 μm, from about 2 μm to about 4 μm, from about 3 μm to about 5 μm, from about 4 μm to about 6 μm, from about 5 μm to about 7 μm, from about 6 μm to about 8 μm, from about 7 μm to about 9 μm, from about 8 μm to about 10 μm. In other aspects, the diameter of the biodegradable microspheres may range from about 1 μm to about 10 μm.

In various other aspects, the composition of active compounds and optional excipients encapsulated within the microspheres includes the active compounds SPP1 and CXCL12. In some aspects, each microsphere encapsulates both SPP1 and CXCL12 in the same microsphere. In other aspects, a first portion of the microspheres loaded into the scaffold contain SPP1 only and a second portion of the loaded microspheres contain CXCL12 only. In various aspects, the SPP1 and the CXCL12 are loaded into the microspheres at any suitable density without limitation. In some aspects, the SPP1 is loaded into the microspheres at a density ranging from about 0.1 μg/ml to about 100 μg/ml. In other aspects, the SPP1 is loaded into the microspheres at a density ranging from about 0.1 μg/ml to about 1 μg/ml, from about 0.5 μg/ml to about 5 μg/ml, from about 1 μg/ml to about 10 μg/ml, from about 5 μg/ml to about 20 μg/ml, from about 10 μg/ml to about 30 μg/ml, from about 20 μg/ml to about 40 μg/ml, from about 30 μg/ml to about 50 μg/ml, from about 40 μg/ml to about 60 μg/ml, from about 50 μg/ml to about 70 μg/ml, from about 60 μg/ml to about 80 μg/ml, from about 70 μg/ml to about 90 μg/ml, from about 80 μg/ml to about 100 μg/ml, from about 90 μg/ml to about 110 μg/ml, from about 100 μg/ml to about 150 μg/ml, from about 125 μg/ml to about 175 μg/ml, and from about 150 μg/ml to about 200 μg/ml.

In some aspects, the CXCL12 is loaded into the microspheres at a density ranging from about 0.1 μg/ml to about 100 μg/ml. In other aspects, the CXCL12 is loaded into the microspheres at a density ranging from about 0.1 μg/ml to about 1 μg/ml, from about 0.5 μg/ml to about 5 μg/ml, from about 1 μg/ml to about 10 μg/ml, from about 5 μg/ml to about 20 μg/ml, from about 10 μg/ml to about 30 μg/ml, from about 20 μg/ml to about 40 μg/ml, from about 30 μg/ml to about 50 μg/ml, from about 40 μg/ml to about 60 μg/ml, from about 50 μg/ml to about 70 μg/ml, from about 60 μg/ml to about 80 μg/ml, from about 70 μg/ml to about 90 μg/ml, and from about 80 μg/ml to about 100 μg/ml.

In various other aspects, the composition of active compounds and optional excipients encapsulated within the microspheres includes one or more additional compounds to modulate the release of the active compounds within the microspheres according to a desired release profile. Non-limiting examples of suitable excipients include heparin.

In various aspects, the sustained release profile is configured to provide direct release of SPP1 and CXCL12 onto the region of the fracture non-union and/or within the gap of the fracture non-union over a preselected duration. In some aspects, the duration of the sustained release profile is at least about 1 week, at least about 2 weeks, at least about 3 weeks, at least about 4 weeks, at least about 5 weeks, at least about 6 weeks, at least about 8 weeks, and at least about 10 weeks. In various aspects, the duration of the sustained release profile ranges from about one week to about 10 weeks. In other aspects, the duration of the sustained release profile ranges from about one week to about 2 weeks, from about 1.5 weeks to about 2.5 weeks, from about 2 weeks to about 3 weeks, from about 2.5 weeks to about 3.5 weeks, from about 3 weeks to about 4 weeks, from about 3.5 weeks to about 4.5 weeks, from about 4 weeks to about 5 weeks, from about 4.5 weeks to about 5.5 weeks, from about 5 weeks to about 7 weeks, from about 6 weeks to about 8 weeks, from about 7 weeks to about 9 weeks, and from about 8 weeks to about 10 weeks.

In various aspects, the formulation may be produced using any suitable method without limitation including, but not limited to, the electrospray method described in the Examples below.

The agents and compositions described herein can be formulated by any conventional manner using one or more pharmaceutically acceptable carriers or excipients as described in, for example, Remington's Pharmaceutical Sciences (A. R. Gennaro, Ed.), 21st edition, ISBN: 0781746736 (2005), incorporated herein by reference in its entirety. Such formulations will contain a therapeutically effective amount of a biologically active agent described herein, which can be in purified form, together with a suitable amount of carrier so as to provide the form for proper administration to the subject.

The term “formulation” refers to preparing a drug in a form suitable for administration to a subject, such as a human. Thus, a “formulation” can include pharmaceutically acceptable excipients, including diluents or carriers.

The term “pharmaceutically acceptable” as used herein can describe substances or components that do not cause unacceptable losses of pharmacological activity or unacceptable adverse side effects. Examples of pharmaceutically acceptable ingredients can be those having monographs in United States Pharmacopeia (USP 29) and National Formulary (NF 24), United States Pharmacopeial Convention, Inc, Rockville, Md., 2005 (“USP/NF”), or a more recent edition, and the components listed in the continuously updated Inactive Ingredient Search online database of the FDA. Other useful components that are not described in the USP/NF, etc. may also be used.

The term “pharmaceutically acceptable excipient,” as used herein, can include any and all solvents, dispersion media, coatings, antibacterial and antifungal agents, isotonic, or absorption delaying agents. The use of such media and agents for pharmaceutically active substances is well known in the art (see generally Remington's Pharmaceutical Sciences (A. R. Gennaro, Ed.), 21st edition, ISBN: 0781746736 (2005)). Except insofar as any conventional media or agent is incompatible with an active ingredient, its use in the therapeutic compositions is contemplated. Supplementary active ingredients can also be incorporated into the compositions.

A “stable” formulation or composition can refer to a composition having sufficient stability to allow storage at a convenient temperature, such as between about 0° C. and about 60° C., for a commercially reasonable period of time, such as at least about one day, at least about one week, at least about one month, at least about three months, at least about six months, at least about one year, or at least about two years.

The formulation should suit the mode of administration. The agents of use with the current disclosure can be formulated by known methods for administration to a subject using several routes which include, but are not limited to, parenteral, pulmonary, oral, topical, intradermal, intratumoral, intranasal, inhalation (e.g., in an aerosol), implanted, intramuscular, intraperitoneal, intravenous, intrathecal, intracranial, intracerebroventricular, subcutaneous, intranasal, epidural, intrathecal, ophthalmic, transdermal, buccal, and rectal. The individual agents may also be administered in combination with one or more additional agents or together with other biologically active or biologically inert agents. Such biologically active or inert agents may be in fluid or mechanical communication with the agent(s) or attached to the agent(s) by ionic, covalent, Van der Waals, hydrophobic, hydrophilic, or other physical forces.

Controlled-release (or sustained-release) preparations may be formulated to extend the activity of the agent(s) and reduce dosage frequency. Controlled-release preparations can also be used to effect the time of onset of action or other characteristics, such as blood levels of the agent, and consequently affect the occurrence of side effects. Controlled-release preparations may be designed to initially release an amount of an agent(s) that produces the desired therapeutic effect, and gradually and continually release other amounts of the agent to maintain the level of therapeutic effect over an extended period of time. In order to maintain a near-constant level of an agent in the body, the agent can be released from the dosage form at a rate that will replace the amount of agent being metabolized or excreted from the body. The controlled-release of an agent may be stimulated by various inducers, e.g., change in pH, change in temperature, enzymes, water, or other physiological conditions or molecules.

Agents or compositions described herein can also be used in combination with other therapeutic modalities, as described further below. Thus, in addition to the therapies described herein, one may also provide to the subject other therapies known to be efficacious for treatment of the disease, disorder, or condition.

Modulation Agents

As described herein, CXCL12 and/or SPP1 expression has been implicated in various diseases, disorders, and conditions. As such, modulation of CXCL12 and/or SPP1 (e.g., modulation of CXCL12 and/or SPP1) can be used for treatment of such conditions. A CXCL12 and/or SPP1 modulation agent can modulate CXCL12 and/or SPP1 response or induce or inhibit CXCL12 and/or SPP1. CXCL12 and/or SPP1 modulation can comprise modulating the expression of CXCL12 and/or SPP1 on cells, modulating the quantity of cells that express CXCL12 and/or SPP1 or modulating the quality of the CXCL12 and/or SPP1 expressing cells.

CXCL12 and/or SPP1 modulation agents can be any composition or method that can modulate CXCL12 and/or SPP1 expression on cells. For example, a CXCL12 and/or SPP1 modulation agent can be an activator, an inhibitor, an agonist, or an antagonist. As another example, the CXCL12 and/or SPP1 modulation can be the result of gene editing.

A CXCL12 and/or SPP1 modulation agent can be a CXCL12 and/or SPP1 antibody (e.g., a monoclonal antibody to CXCL12 and/or SPP1).

A CXCL12 and/or SPP1 modulating agent can be an agent that induces or inhibits progenitor cell differentiation into CXCL12 and/or SPP1 expressing cells.

Therapeutic Methods

Also provided is a process of treating, preventing, or reversing fracture nonunions in a subject in need of administration of a therapeutically effective amount of CXCL12 and/or SPP1, so as to heal fracture nonunions.

In various aspects, the method includes providing a formulation comprising a flexible biodegradable scaffold loaded with CXCL12 and SPP1 as described herein, and applying the formulation over a region of the fracture non-union and/or within a gap of the fracture non-union. After application of the formulation, CXCL12 and SPP1 are locally released to the region of the fracture non-union and/or within a gap of the fracture non-union according to a predetermined release profile defined by various characteristics of the scaffold and CXCL12/SPP1-loaded microspheres, as described herein. In various aspects, the therapeutically effective amounts of CXCL12 and SPPI locally released to the region of the fracture non-union may be adjusted by modulating the number of scaffold layers applied to the region of the fracture non-union.

Methods described herein are generally performed on a subject in need thereof. A subject in need of the therapeutic methods described herein can be a subject having, diagnosed with, suspected of having, or at risk for developing fracture nonunions. A determination of the need for treatment will typically be assessed by a history, physical exam, or diagnostic tests consistent with the disease or condition at issue. Diagnosis of the various conditions treatable by the methods described herein is within the skill of the art. The subject can be an animal subject, including a mammal, such as horses, cows, dogs, cats, sheep, pigs, mice, rats, monkeys, hamsters, guinea pigs, and humans or chickens. For example, the subject can be a human subject.

Generally, a safe and effective amount of CXCL12 and/or SPP1 is, for example, an amount that would cause the desired therapeutic effect in a subject while minimizing undesired side effects.

According to the methods described herein, administration can be parenteral, pulmonary, oral, topical, intradermal, intramuscular, intraperitoneal, intravenous, intratumoral, intrathecal, intracranial, intracerebroventricular, subcutaneous, intranasal, epidural, ophthalmic, buccal, or rectal administration.

When used in the treatments described herein, a therapeutically effective amount of CXCL12 and/or SPP1 can be employed in pure form or, where such forms exist, in pharmaceutically acceptable salt form and with or without a pharmaceutically acceptable excipient. For example, the compounds of the present disclosure can be administered, at a reasonable benefit/risk ratio applicable to any medical treatment, in a sufficient amount to heal fracture nonunions.

The amount of a composition described herein that can be combined with a pharmaceutically acceptable carrier to produce a single dosage form will vary depending upon the subject or host treated and the particular mode of administration. It will be appreciated by those skilled in the art that the unit content of agent contained in an individual dose of each dosage form need not in itself constitute a therapeutically effective amount, as the necessary therapeutically effective amount could be reached by administration of a number of individual doses.

Toxicity and therapeutic efficacy of compositions described herein can be determined by standard pharmaceutical procedures in cell cultures or experimental animals for determining the LD₅₀ (the dose lethal to 50% of the population) and the ED₅₀, (the dose therapeutically effective in 50% of the population). The dose ratio between toxic and therapeutic effects is the therapeutic index that can be expressed as the ratio LD₅₀/ED₅₀, where larger therapeutic indices are generally understood in the art to be optimal.

The specific therapeutically effective dose level for any particular subject will depend upon a variety of factors including the disorder being treated and the severity of the disorder; activity of the specific compound employed; the specific composition employed; the age, body weight, general health, sex and diet of the subject; the time of administration; the route of administration; the rate of excretion of the composition employed; the duration of the treatment; drugs used in combination or coincidental with the specific compound employed; and like factors well known in the medical arts (see e.g., Koda-Kimble et al. (2004) Applied Therapeutics: The Clinical Use of Drugs, Lippincott Williams & Wilkins, ISBN 0781748453; Winter (2003) Basic Clinical Pharmacokinetics, 4th ed., Lippincott Williams & Wilkins, ISBN 0781741475; Sharqel (2004) Applied Biopharmaceutics & Pharmacokinetics, McGraw-Hill/Appleton & Lange, ISBN 0071375503). For example, it is well within the skill of the art to start doses of the composition at levels lower than those required to achieve the desired therapeutic effect and to gradually increase the dosage until the desired effect is achieved. If desired, the effective daily dose may be divided into multiple doses for purposes of administration. Consequently, single dose compositions may contain such amounts or submultiples thereof to make up the daily dose. It will be understood, however, that the total daily usage of the compounds and compositions of the present disclosure will be decided by an attending physician within the scope of sound medical judgment.

Again, each of the states, diseases, disorders, and conditions, described herein, as well as others, can benefit from compositions and methods described herein. Generally, treating a state, disease, disorder, or condition includes preventing, reversing, or delaying the appearance of clinical symptoms in a mammal that may be afflicted with or predisposed to the state, disease, disorder, or condition but does not yet experience or display clinical or subclinical symptoms thereof. Treating can also include inhibiting the state, disease, disorder, or condition, e.g., arresting or reducing the development of the disease or at least one clinical or subclinical symptom thereof. Furthermore, treating can include relieving the disease, e.g., causing regression of the state, disease, disorder, or condition or at least one of its clinical or subclinical symptoms. A benefit to a subject to be treated can be either statistically significant or at least perceptible to the subject or to a physician.

Administration of CXCL12 and/or SPP1 can occur as a single event or over a time course of treatment. For example, CXCL12 and/or SPP1 can be administered daily, weekly, bi-weekly, or monthly. For treatment of acute conditions, the time course of treatment will usually be at least several days. Certain conditions could extend treatment from several days to several weeks. For example, treatment could extend over one week, two weeks, or three weeks. For more chronic conditions, treatment could extend from several weeks to several months or even a year or more.

Treatment in accord with the methods described herein can be performed prior to, concurrent with, or after conventional treatment modalities for healing fracture nonunions.

A CXCL12 and/or SPP1 can be administered simultaneously or sequentially with another agent, such as an antibiotic, an anti-inflammatory, or another agent. For example, a CXCL12 and/or SPP1 can be administered simultaneously with another agent, such as an antibiotic or an anti-inflammatory. Simultaneous administration can occur through administration of separate compositions, each containing one or more of a CXCL12 and/or SPP1, an antibiotic, an anti-inflammatory, or another agent. Simultaneous administration can occur through administration of one composition containing two or more of a CXCL12 and/or SPP1, an antibiotic, an anti-inflammatory, or another agent. A CXCL12 and/or SPP1 can be administered sequentially with an antibiotic, an anti-inflammatory, or another agent. For example, a CXCL12 and/or SPP1 can be administered before or after administration of an antibiotic, an anti-inflammatory, or another agent.

Administration

Agents and compositions described herein can be administered according to methods described herein in a variety of means known to the art. The agents and composition can be used therapeutically either as exogenous materials or as endogenous materials. Exogenous agents are those produced or manufactured outside of the body and administered to the body. Endogenous agents are those produced or manufactured inside the body by some type of device (biologic or other) for delivery within or to other organs in the body.

As discussed above, administration can be parenteral, pulmonary, oral, topical, intradermal, intratumoral, intranasal, inhalation (e.g., in an aerosol), implanted, intramuscular, intraperitoneal, intravenous, intrathecal, intracranial, intracerebroventricular, subcutaneous, intranasal, epidural, intrathecal, ophthalmic, transdermal, buccal, and rectal.

Agents and compositions described herein can be administered in a variety of methods well known in the arts. Administration can include, for example, methods involving oral ingestion, direct injection (e.g., systemic or stereotactic), implantation of cells engineered to secrete the factor of interest, drug-releasing biomaterials, polymer matrices, gels, permeable membranes, osmotic systems, multilayer coatings, microparticles, implantable matrix devices, mini-osmotic pumps, implantable pumps, injectable gels and hydrogels, liposomes, micelles (e.g., up to 30 μm), nanospheres (e.g., less than 1 μm), microspheres (e.g., 1-100 μm), reservoir devices, a combination of any of the above, or other suitable delivery vehicles to provide the desired release profile in varying proportions. Other methods of controlled-release delivery of agents or compositions will be known to the skilled artisan and are within the scope of the present disclosure.

Delivery systems may include, for example, an infusion pump that may be used to administer the agent or composition in a manner similar to that used for delivering insulin or chemotherapy to specific organs or tumors. Typically, using such a system, an agent or composition can be administered in combination with a biodegradable, biocompatible polymeric implant that releases the agent over a controlled period of time at a selected site. Examples of polymeric materials include polyanhydrides, polyorthoesters, polyglycolic acid, polylactic acid, polyethylene vinyl acetate, and copolymers and combinations thereof. In addition, a controlled release system can be placed in proximity of a therapeutic target, thus requiring only a fraction of a systemic dosage.

Agents can be encapsulated and administered in a variety of carrier delivery systems. Examples of carrier delivery systems include microspheres, hydrogels, polymeric implants, smart polymeric carriers, and liposomes (see generally, Uchegbu and Schatzlein, eds. (2006) Polymers in Drug Delivery, CRC, ISBN-10: 0849325331). Carrier-based systems for molecular or biomolecular agent delivery can: provide for intracellular delivery; tailor biomolecule/agent release rates; increase the proportion of biomolecule that reaches its site of action; improve the transport of the drug to its site of action; allow colocalized deposition with other agents or excipients; improve the stability of the agent in vivo; prolong the residence time of the agent at its site of action by reducing clearance; decrease the nonspecific delivery of the agent to nontarget tissues; decrease irritation caused by the agent; decrease toxicity due to high initial doses of the agent; alter the immunogenicity of the agent; decrease dosage frequency, improve taste of the product; or improve shelf life of the product.

Screening

Also provided are methods for screening.

The subject methods find use in the screening of a variety of different candidate molecules (e.g., potentially therapeutic candidate molecules). Candidate substances for screening according to the methods described herein include, but are not limited to, fractions of tissues or cells, nucleic acids, polypeptides, siRNAs, antisense molecules, aptamers, ribozymes, triple helix compounds, antibodies, and small (e.g., less than about 2000 mw, or less than about 1000 mw, or less than about 800 mw) organic molecules or inorganic molecules including but not limited to salts or metals.

Candidate molecules encompass numerous chemical classes, for example, organic molecules, such as small organic compounds having a molecular weight of more than 50 and less than about 2,500 Daltons. Candidate molecules can comprise functional groups necessary for structural interaction with proteins, particularly hydrogen bonding, and typically include at least an amine, carbonyl, hydroxyl or carboxyl group, and usually at least two of the functional chemical groups. The candidate molecules can comprise cyclical carbon or heterocyclic structures and/or aromatic or polyaromatic structures substituted with one or more of the above functional groups.

A candidate molecule can be a compound in a library database of compounds. One of skill in the art will be generally familiar with, for example, numerous databases for commercially available compounds for screening (see e.g., ZINC database, UCSF, with 2.7 million compounds over 12 distinct subsets of molecules; Irwin and Shoichet (2005) J Chem Inf Model 45, 177-182). One of skill in the art will also be familiar with a variety of search engines to identify commercial sources or desirable compounds and classes of compounds for further testing (see e.g., ZINC database; eMolecules.com; and electronic libraries of commercial compounds provided by vendors, for example: ChemBridge, Princeton BioMolecular, Ambinter SARL, Enamine, ASDI, Life Chemicals etc.).

Candidate molecules for screening according to the methods described herein include both lead-like compounds and drug-like compounds. A lead-like compound is generally understood to have a relatively smaller scaffold-like structure (e.g., molecular weight of about 150 to about 350 kD) with relatively fewer features (e.g., less than about 3 hydrogen donors and/or less than about 6 hydrogen acceptors; hydrophobicity character xlogP of about −2 to about 4) (see e.g., Angewante (1999) Chemie Int. ed. Engl. 24, 3943-3948). In contrast, a drug-like compound is generally understood to have a relatively larger scaffold (e.g., molecular weight of about 150 to about 500 kD) with relatively more numerous features (e.g., less than about 10 hydrogen acceptors and/or less than about 8 rotatable bonds; hydrophobicity character xlogP of less than about 5) (see e.g., Lipinski (2000) J. Pharm. Tox. Methods 44, 235-249). Initial screening can be performed with lead-like compounds.

When designing a lead from spatial orientation data, it can be useful to understand that certain molecular structures are characterized as being “drug-like”. Such characterization can be based on a set of empirically recognized qualities derived by comparing similarities across the breadth of known drugs within the pharmacopoeia. While it is not required for drugs to meet all, or even any, of these characterizations, it is far more likely for a drug candidate to meet with clinical successful if it is drug-like.

Several of these “drug-like” characteristics have been summarized into the four rules of Lipinski (generally known as the “rules of fives” because of the prevalence of the number 5 among them). While these rules generally relate to oral absorption and are used to predict bioavailability of compound during lead optimization, they can serve as effective guidelines for constructing a lead molecule during rational drug design efforts such as may be accomplished by using the methods of the present disclosure.

The four “rules of five” state that a candidate drug-like compound should have at least three of the following characteristics: (i) a weight less than 500 Daltons; (ii) a log of P less than 5; (iii) no more than 5 hydrogen bond donors (expressed as the sum of OH and NH groups); and (iv) no more than 10 hydrogen bond acceptors (the sum of N and O atoms). Also, drug-like molecules typically have a span (breadth) of between about 8 Å to about 15 Å.

Kits

Also provided are kits. Such kits can include an agent or composition described herein and, in certain embodiments, instructions for administration. Such kits can facilitate performance of the methods described herein. When supplied as a kit, the different components of the composition can be packaged in separate containers and admixed immediately before use. Such packaging of the components separately can, if desired, be presented in a pack or dispenser device which may contain one or more unit dosage forms containing the composition. The pack may, for example, comprise metal or plastic foil such as a blister pack. Such packaging of the components separately can also, in certain instances, permit long-term storage without losing activity of the components.

Kits may also include reagents in separate containers such as, for example, sterile water or saline to be added to a lyophilized active component packaged separately. For example, sealed glass ampules may contain a lyophilized component and in a separate ampule, sterile water, sterile saline each of which has been packaged under a neutral non-reacting gas, such as nitrogen. Ampules may consist of any suitable material, such as glass, organic polymers, such as polycarbonate, polystyrene, ceramic, metal or any other material typically employed to hold reagents. Other examples of suitable containers include bottles that may be fabricated from similar substances as ampules, and envelopes that may consist of foil-lined interiors, such as aluminum or an alloy. Other containers include test tubes, vials, flasks, bottles, syringes, and the like. Containers may have a sterile access port, such as a bottle having a stopper that can be pierced by a hypodermic injection needle.

Other containers may have two compartments that are separated by a readily removable membrane that upon removal permits the components to mix. Removable membranes may be glass, plastic, rubber, and the like.

In certain embodiments, kits can be supplied with instructional materials. Instructions may be printed on paper or other substrate, and/or may be supplied as an electronic-readable medium or video. Detailed instructions may not be physically associated with the kit; instead, a user may be directed to an Internet web site specified by the manufacturer or distributor of the kit.

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

All publications, patents, patent applications, and other references cited in this application are incorporated herein by reference in their entirety for all purposes to the same extent as if each individual publication, patent, patent application or other reference was specifically and individually indicated to be incorporated by reference in its entirety for all purposes. Citation of a reference herein shall not be construed as an admission that such is prior art to the present disclosure.

Having described the present disclosure in detail, it will be apparent that modifications, variations, and equivalent embodiments are possible without departing the scope of the present disclosure defined in the appended claims. Furthermore, it should be appreciated that all examples in the present disclosure are provided as non-limiting examples.

### Examples

The following non-limiting examples are provided to further illustrate the present disclosure. It should be appreciated by those of skill in the art that the techniques disclosed in the examples that follow represent approaches the inventors have found function well in the practice of the present disclosure, and thus can be considered to constitute examples of modes for its practice.

However, those of skill in the art should, in light of the present disclosure, appreciate that many changes can be made in the specific embodiments that are disclosed and still obtain a like or similar result without departing from the spirit and scope of the present disclosure.

Atrophic fracture nonunion poses a significant clinical problem with limited therapeutic interventions. A unique nonunion model with high clinical relevance was developed, using serum transfer-induced rheumatoid arthritis (RA) in mice. Arthritic mice displayed fracture nonunion with an absence of fracture callus, diminished angiogenesis and fibrotic scar tissue formation, leading to failure of biomechanical properties, which represented the major manifestations of atrophic nonunion in the clinic. Mechanistically, we demonstrated that the angiogenesis defect observed in RA mice was due to downregulation of SPP1 and CXCL12 in chondrocytes, as evidenced by restored angiogenesis upon SPP1 and CXCL12 treatment in vitro.

### Methods

Animals. Male C57BL/6J wild type mice (Jackson Laboratory #000664) and Relb-Luc (NF-κB-GFP-Luciferase) reporter mice (Jackson Laboratory (#027529) were purchased from a commercial vendor. Jackson Laboratory (#000664). The Relb-Luc (NF-κB-GFP-Luciferase) reporter mice were used to visualize the NF-κB activity in vivo. Systemic inflammation was induced in 12-week-old mice via intraperitoneal injection of 100 μl arthritic K/B×N serum at day 0 and day 3, and systemic inflammation was maintained by continuous injection every five days until the endpoint of experiments. Control mice were injected with phosphate-buffered saline (PBS). Bony fracture procedures were performed on right tibiae as described in Wang et al. 2018, J Bone Miner Res 33, 283-297, the contents of which are incorporated by reference in their entirety.

Bioluminescence imaging. PBS and K/B×N serum treated Relb-Luc reporter mice received 150 mg/kg of luciferin and were continuously monitored by the IVIS 50 imaging system (Xenogen/PerkinElmer) every three days for 15 days. The luminescent intensities of the right hind legs were analyzed using Living Image 3.0 software (Xenogen).

Histological analyses of fracture callus. The fractured tibiae were collected for histological analyses at 7, 10, 14, and 21 days post-fracture (dpf). Following 10% neutral buffered formalin (NBF) fixation and decalcification by 14% ethylenediaminetetraacetic acid (EDTA), the fractured tibiae were embedded in paraffin, sectioned in 5 μm and stained by Alcian blue/Hematoxylin/Orange G (ABH/OG) and tartrate-resistant acid phosphatase (TRAP) stainings. Cartilage and bone area were measured using ImageJ software (Wayne Rasband) based on ABH/OG staining. Osteoclast surface per bone surface (Oc. S/BS) were measured and calculated based on TRAP staining on 14 dpf fracture callus. IHC staining for type III collagen (1:500, Abcam, #ab7778), SPP1 (1:50, Abcam, #ab8448), and CXCL12 (1:50, LSBio, #LS-B2437) were performed via proteinase K antigen retrieval and DAB (3,3′-Diaminobenzidine, Vector Laboratories, #SK-4100) mediated colorimetric development. Immunofluorescence staining for Endomucin (1:100, Santa Cruz, #sc-65495) was performed via proteinase K antigen retrieval and Alexa Fluor 488 antibody labeling kit (ThermoFisher, #A20181) mediated fluorescent development. Quantification of vessel number was based on the Endomucin immunofluorescence staining on the central fracture area (800 μm×600 μm) and the relative vessel count was calculated by normalization of the experimental group to the control group.

microCT analyses. The fractured tibiae were collected at 10, 14, and 21 dpf and examined by microCT scanner (VivaCT 40, Scanco) with the parameters of 55 kV, 145 μA, and 300 ms integration time. 3D images were generated using the Scanco software. Quantification of the bone volume (BV) and bone volume to total volume (BV/TV) was based on 600 slices centered on the fracture site. The relative BV and BV/TV were calculated by normalization of the experimental group to the control group.

In vivo angiogenesis analysis. The animal was perfused with Microfil MV-122 (Flowtech, #MV-122), a lead chromate-based contrast agent and then the vascular structure surrounding the fracture site in the tibia was examined by microCT with the parameters of 55 kV, 145 μA, and 300 ms integration time. 3D images were generated using the Scanco software. In this experiment, we analyzed the center of the fracture area where the atrophic region was located and the surrounding region. Quantification of the vessel number was based on 20 slices centered on the fracture site. The relative vessel count was calculated by normalization of the experimental group to the control group.

Biomechanical torsion testing. The fractured tibiae were harvested at 28 dpf and the ends were secured with methacrylate (MMA) into 1.2 cm long cylinders to position the fracture site in the middle. The fracture tibiae were tested in torsion using the custom LabVIEW (National Instruments) program until failure. The maximum torque and displacement at maximum torque were recorded and processed by a custom MATLAB 2017b program (Mathworks).

Primary chondrocyte isolation and culture. Primary chondrocytes were isolated from ribcages of neonatal C57BL/6J pups. The ribcages were dissected without soft tissue, followed by 2 mg/ml pronase (Millipore Sigma, #10165921001) and 3 mg/ml collagenase D (Millipore Sigma, #11088866001) digestion with agitation. The remaining sterna were further digested with 3 mg/ml collagenase D for 4 to 6 hours. Chondrocytes were collected and cultured in DMEM with 10% FBS. Following 48 hours treatment of vehicle and 1 ng/ml IL-1β, the cultured medium was collected for the angiogenic protein array and HUVEC (human umbilical vein endothelial cell) angiogenesis assay.

Primary osteoblast isolation and culture. Primary osteoblasts were isolated from calvaries of neonatal C57BL/6J pups. Briefly, after removing the soft tissue and sutures, calvaries were digested with 0.1% dispase (Millipore Sigma, #D4693) and 0.1% collagenase P (Millipore Sigma, #11249002001). Osteoblast released from mouse calvaries were collected and stimulated with 50 μg/ml ascorbic acid (Millipore Sigma, #57803) for 3 days for maturation. Following 48 hours of vehicle and 1 ng/ml IL-1β treatments on mature osteoblasts, the cultured media were collected for the angiogenic protein array and HUVEC angiogenesis assay.

Angiogenic protein array. Proteome Profiler Mouse Angiogenesis Array Kit (R&D, #ARY015) was used to examine the expression of angiogenic factors in medium collected from chondrocyte and osteoblast cultures according to the manufacturer's protocol. Bio-rad ChemiDoc imaging system was used to visualize and quantify the array signals.

In vitro angiogenesis assay. In the tube formation assay, 4×10⁴ HUVEC cells were seeded and cultured in 96 well plates precoated with Matrigel (Corning, #356237) for 12 hours. The formed tube numbers were examined by Image J software. In the migration assay, the media collected from chondrocyte cultures were added to the bottom chamber. 4×10⁴ HUVEC cells were seeded and cultured in the upper chamber of 24 well transwell plate (Millipore Sigma, #0L53422) with serum-free medium for 12 hours. The number of migrated HUVEC cells was counted based on the crystal violet staining. 100 ng/ml of CXCL12 (Roche, #460-SD-010) and 500 ng/ml of SPP1 (Roche, #441-OP-050) were also supplemented respectively in the chondrocyte culture medium for HUVEC tube formation and migration assays. HUVEC cell proliferation and apoptosis under cultured medium from vehicle and IL-1β treated chondrocytes were assessed by Roche Cell Proliferation ELISA Kit (Roche, #11647229001) and Cell Death Detection ELISA Kit (Roche, #11774425001) respectively.

Fabrication of PCL scaffold with growth factors. The PCL scaffold was fabricated with PLGA microspheres containing SPP1 and CXCL12 via a co-axial electrospraying and electrospinning method. Briefly, the PCL (Millipore Sigma, #440744) was dissolved in chloroform (Millipore Sigma, #372978) with a final concentration of 5% and stored in syringe pump A (PCL solution in FIG. 4A). 5% PLGA (Millipore Sigma, #P2191) was dissolved in methylene chloride (Millipore Sigma, #M1550000) and stored at syringe pump B (PLGA solution in FIG. 4A). A mixture of growth factors that included 100 μg/ml SPP1 (Peprotec, #120-35) and 20 μg/ml CXCL12 (Peprotec, #300-28B) were mixed with 0.5% gelatin (Millipore Sigma, #G6650) in syringe pump C (Growth factor solution in FIG. 4A). PCL was injected at the rate of 5 ml/h onto a collecting mandrel positioned 20 cm away from the exit port of syringe pump A. The PCL solution was charged at +17 kV while the collecting mandrel was charged at −10 kV. The diameter of the collecting mandrel was 13 cm and the rotation rate was 800 rpm. The PLGA solution was sprayed at the rate of 1 ml/h into the exterior aisle of a coaxial exit port and the solution containing growth factors was sprayed at the rate of 0.5 ml/h into the interior aisle of the coaxial exit port simultaneously. The inner diameter of the coaxial exit port was 0.70 mm and the outer diameter was 1.65 mm. The PLGA and growth factor solution were delivered by the coaxial exit port at a charge of +20 kV, while the collecting mandrel was charged at −10 kV. The distance between the coaxial electrospray needle and the collector was 30 cm. Four types of scaffolds were fabricated, i.e., scaffold with no growth factor, scaffold with SPP1 alone, scaffold with CXCL12 alone and scaffold with both SPP1 and CXCL12.

Scaffold characterization. We mixed PLGA solution with 10 μg/ml Rhodamine B (Millipore Sigma, #83689) and the mixed growth factor solution was mixed with 50 mg/ml Hoechst (Millipore Sigma, #63493). The core-shell structure was examined by Zeiss LSM880 confocal microscope. The scaffolds containing SPP1 and CXCL12 were sputter-coated with gold. The fiber structure and microsphere distribution were examined by a scanning electron microscope.

The mechanical properties of the PCL scaffold were examined by tensile test. Briefly, the PCL scaffold with 150 μm thickness was cut into 15 mm in length and 1 mm in width. The 100 N load cell and tensile displacement rate of 0.5 mm/min were applied to the scaffold while recording force and the displacement of the PCL scaffold sample. Tensile stress and strain were calculated and Young's modulus was determined in the elastic deformation region of the stress-strain curve.

Release profile of growth factors from PCL scaffolds. 50 mg PCL scaffolds containing growth factors (SPP1 and CXCL12) were placed into 1 ml phosphate-buffered saline (PBS, HyClone, #SH30013.0.3) supplemented with 1% penicillin and streptomycin (ThermoFisher, #15140122), and incubated at 37° C. for 4 weeks. The PBS was collected at predetermined time points, and an equal volume of fresh PBS was added. The collected PBS solution was subjected to an ELISA assay to determine the concentration of SPP1 and CXCL12 according to the manufacturers' protocol (R&D Systems, #DOST00 for SPP1; Peprotec, #900-M92 for CXCL12).

In vitro bioactivity assay of PCL scaffolds. PCL scaffold was placed on the bottom of a 24-well plate. 500 μl of collagen gel (Corning, #354236) was cast onto the top of the scaffold, followed by seeding CM-Dil (Invitrogen, #C7000) labeled HUVEC cells at a density of 1×10⁵ cells/ml. After 5 days of culture, the constructs were fixed by 4% paraformaldehyde solution (ThermoFisher, #AAJ19943K2). The cells were imaged by a confocal microscope. Z-stack images were taken in the 20 μm thickness. HUVEC cell migration and lumen formation were quantified based on the constructed 3D images.

Real-time qPCR. 4 mm samples of fracture callus were isolated from control and RA mice and homogenized for RNA extraction using RNeasy Mini Kit (Qiagen, #74106). cDNA synthesis and real-time qPCR was performed following the manufacturers' protocol. Primer sequences for ll1b, ll6, ll10, Tnfa, Spp1, Cxcl12, and Actb were detailed in Table 1.

TABLE 1
Primer Sequences for qPCR

| Genes | SEQ ID NO | Sequence |
|---|---|---|
| 111 b | 1 | 5'-GCA ACT GTT CCT GAA CTC AAC T-3' |
| | 2 | 5'-ATC TTT TGG GGT CCG TCA ACT-3' |
| 116 | 3 | 5'-TCC AGT TGC CTT CTT GGG AC-3' |
| | 4 | 5'-GTA CTC CAG AAG ACC AGA GG-3' |
| 1110 | 5 | 5'-GCT CTT ACT GAC TGG CAT GAG-3' |
| | 6 | 5'-CGC AGC TCT AGG AGC TAG TG-3' |
| Tnfa | 7 | 5'-CAC ACT CAG ATC ATC TTC TCA A-3' |
| | 8 | 5'-AGT AGA CAA GGT ACA ACC CAT C-3' |
| Spp1 | 9 | 5'-TCG TCA TCA TCG TCG TCC A-3' |
| | 10 | 5'-AGA ATG CTG TGT CCT CTG AAG-3' |
| Cxcl12 | 11 | 5'-GAC TCA CAC CTC TCA CAT CTT G-3' |
| | 12 | 5'-GTG CCC TTC AGA TTG TTG C-3' |
| Actb | 13 | 5'-AGA TGT GGA TCA GCA AGC AG-3' |
| | 14 | 5'-GCG CAA GTT AGG TTT TGT CA-3' |

Western blot. Primary chondrocytes were seeded at a density of 1×10⁶ cells/well in a 6-well plate. The wells were treated with vehicle, 20 ng/ml IL-6 (R&D Systems, #406-ML-005) and 20 ng/ml TNFα (R&D Systems, #410-MT-010) for 48 hours. Cell lysates were separated by SDS-polyacrylamide gel and were examined by following antibodies, SPP1 (1:1000, Abcam, #ab8448), CXCL12 (1:1000, LSBio, #LS-B2437), and 6-actin (1:4000, Sigma, #2228).

Statistics. Statistical analyses were performed using Graph Pad Prism. A two-tailed student's t-test was used to determine the significance between the two groups. Comparisons among multiple groups were performed using two-way ANOVA followed by the Tukey test. All data were presented as the mean±SD of at least three independent experiments. p<0.05 was considered statistical y significant.

### Example 1: Elevated Inflammation Leads to Fracture Nonunion in RA Mice

Despite the growing knowledge of atrophic nonunion from animal models, fracture nonunion remains an exceedingly challenging clinical problem with limited and mainly invasive therapeutic interventions. To date, the atrophic nonunion models available for mechanistic and therapeutic studies primarily rely on critical size bone defect and removal of periosteum, which are particularly valuable to delineate the effect of periosteum tissue and progenitor cell differentiation on disease initiation and progression. Nevertheless, these animal models lack clinical relevance and rarely reflect the clinical scenario, since atrophic nonunion is more prevalent in patients experiencing chronic inflammatory conditions, including diabetes and rheumatoid arthritis (RA).

The rising prevalence of inflammatory disease world-wide, especially RA, is associated with debilitating co-morbidities and clinical complications, including delayed fracture union and nonunions. In order to examine the effect of inflammation/RA on the fracture repair, we generated the RA mice via repeated K/B×N serum intraperitoneal (i.p.) administration every 5 days to maintain the systemic inflammation. Given the fact that the transcription factor NF-κB is the principal mediator and surrogate of inflammatory responses, we examined its activity using NF-κB/RelA-Luc reporter mice subjected to RA condition.

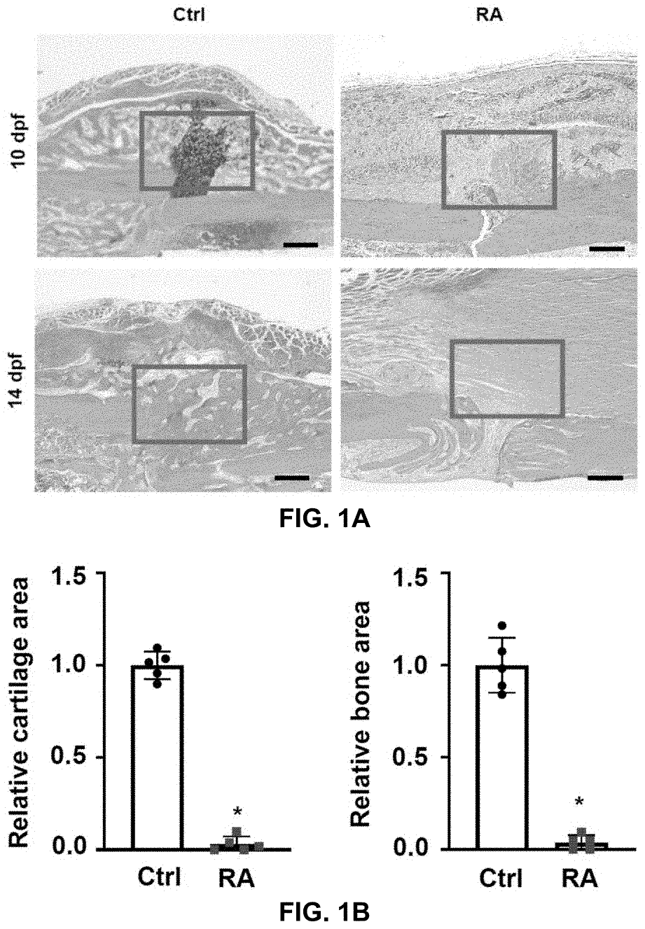

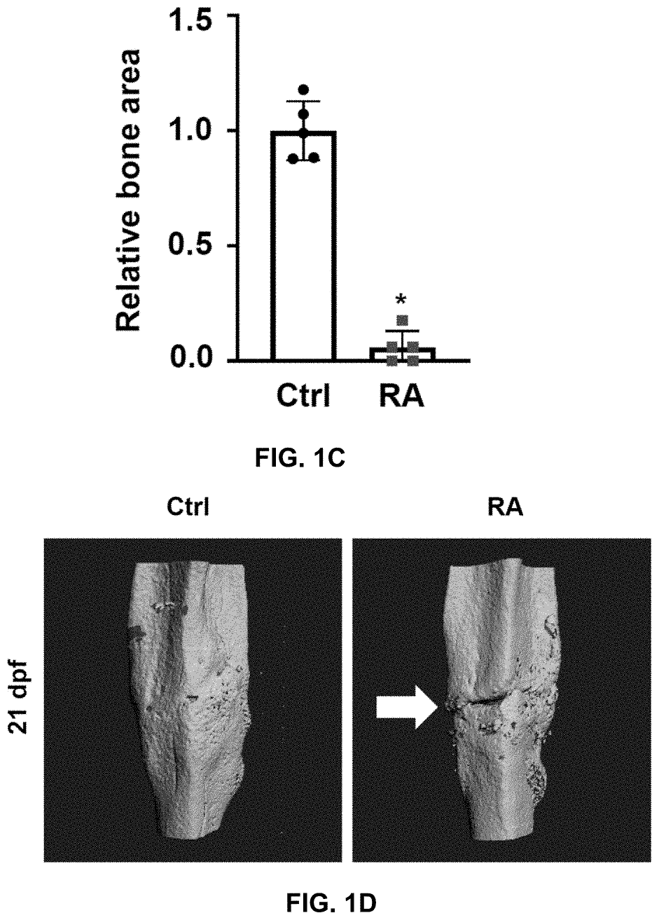

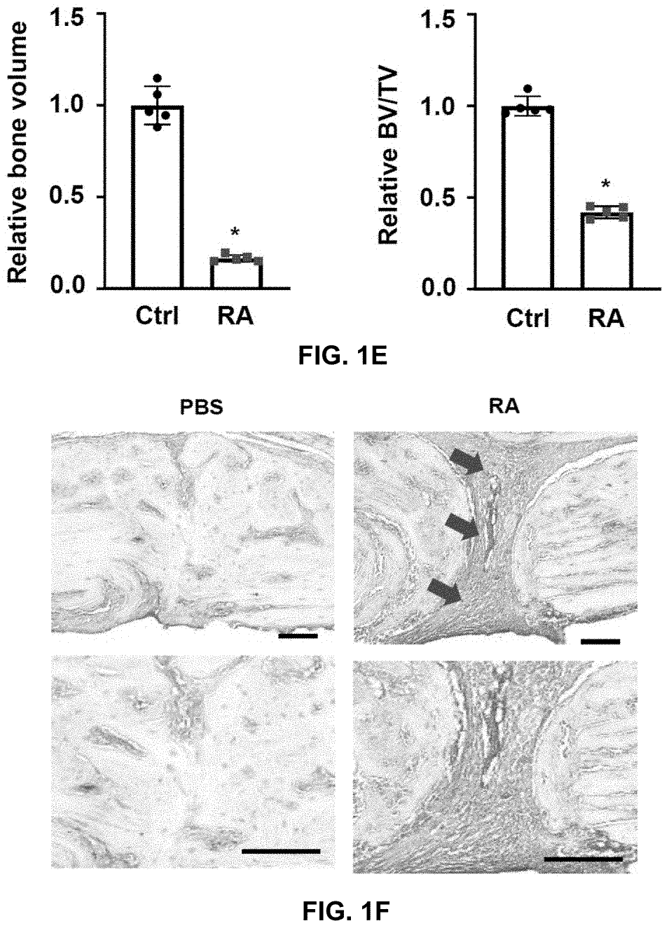

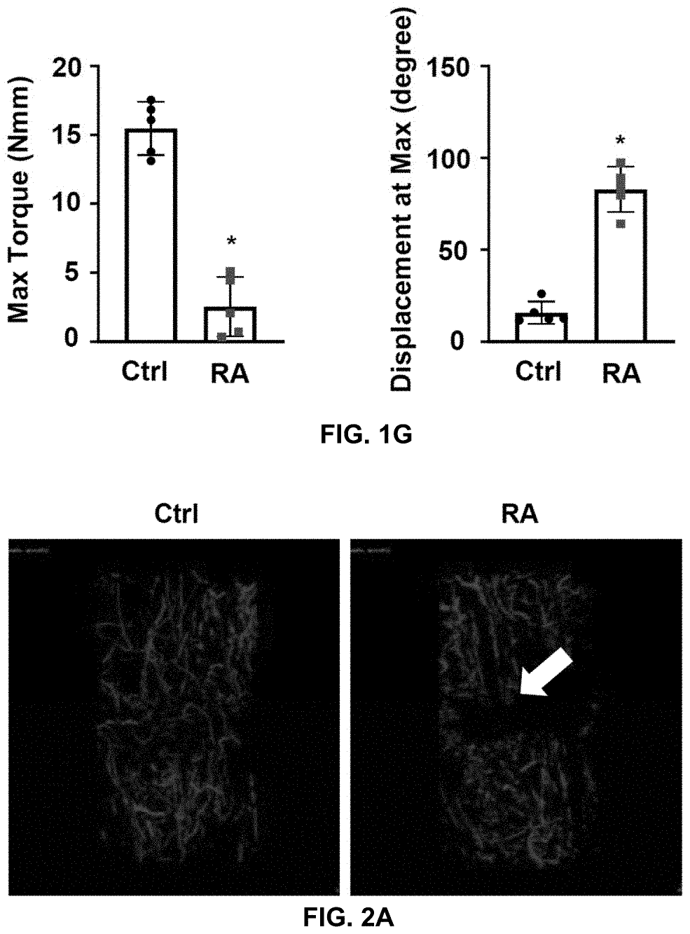

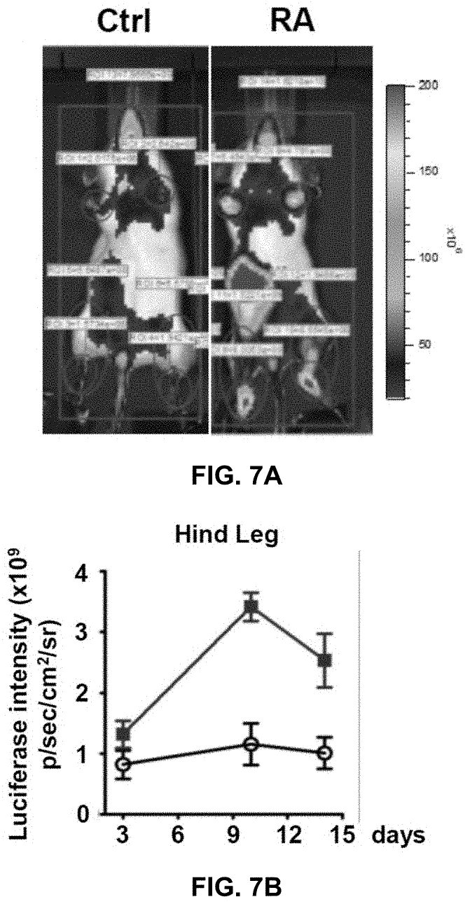

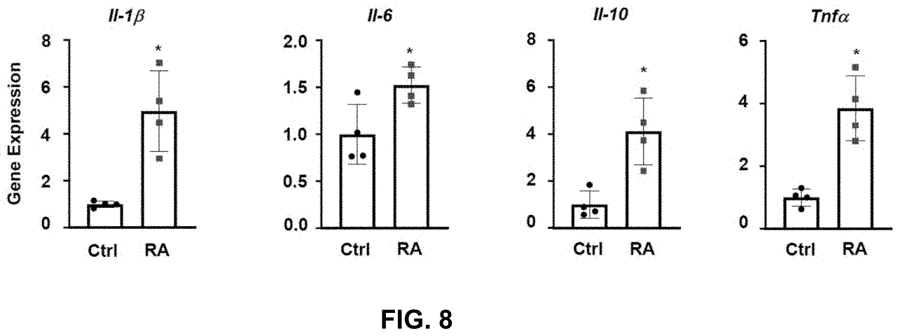

Consistent with the expected increase in inflammation, we confirmed that the RA mice displayed significantly higher luciferase activity, especially in limbs, compared to the control group (FIG. 7A). Importantly, elevated inflammation peaked at 10 days after serum administration and was maintained at least up to 15 days (FIG. 7B). Clinical reports and rodent studies have shown that elevated inflammatory cytokines are the major factors contributing to RA- related co-morbidities. Indeed, we show that mRNA expression levels of several inflammatory cytokines, including ll1β, ll6, ll10, and Tnfa, were significantly elevated in the fracture callus of the RA mice (FIG. 8). Importantly, compared to the control mice, the RA mice developed impaired fracture repair with diminished cartilaginous and bony callus formation reflected by histological analyses of 10 and 14 days post-fracture (dpf) tibae (FIG. 1A). Consistent with histology, quantitative histomorphometry confirmed minimal cartilage template and newly formed woven bone in 10 and 14 dpf RA callus (FIGS. 1B and 1C).

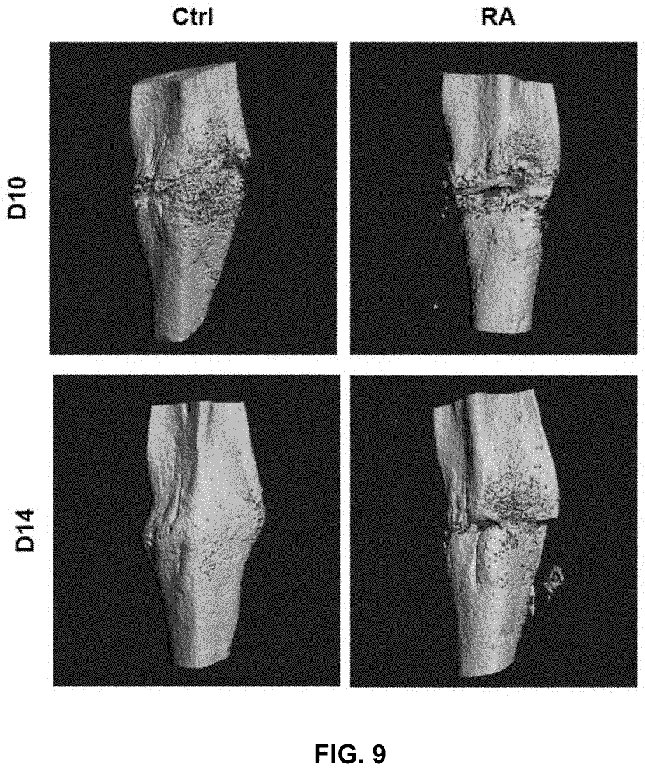

We then performed microCT to further examine the mineralized callus in the control and RA mice. We found a gradual increase in new bone formation around the fracture area by 14 dpf in the control mice. The fracture gap in the control mice was closed at 21 dpf with robust bone formation and complete healing. In contrast, the fracture gap persisted until 21 dpf in the RA mice as shown by microCT, with significantly reduced newly formed bone tissue and bone volume over total volume (BV/TV) (FIGS. 1D, 1E, and 9). Notably, Collagen III positive fibrotic tissue was present in the center of RA fracture callus instead of woven bone tissue in the control fracture, suggesting an atrophic nonunion fracture in the RA mice (FIG. 1F).

Finally, we performed the torsion testing to evaluate the mechanical properties of 28 dpf tibae as the ultimate indication of fracture repair outcome in the control and RA mice. Compared to the control fractured tibae, the RA fractured tibae displayed significantly lower maximum torque (77% reduction) with a larger displacement angle, indicating poor bone strength and rigidity of newly formed bone in the RA mice (FIG. 1G). Altogether, these data confirmed that under RA condition, mice displayed systemic inflammation mediated by cytokines, and developed fracture nonunion.

The results of these experiments demonstrated the development of atrophic nonunion of bone fractures under the chronic inflammation induced by K/B×N serum in the RA mouse model with an absence of fracture callus, as well as diminished angiogenesis. Despite a large amount of evidence on the role of the NF-κB pathway in fracture studies, the mechanisms by which pathologic inflammation adversely affects angiogenesis during fracture healing were previously unknown. Specifically, NF-κb reporter mice confirmed that elevated inflammation was exhibited in the RA mice particularly in the hind limbs that was maintained at least up to 15 days. The expression of inflammatory factors was also induced locally in the fracture callus of RA mice.

Furthermore, the RA mice displayed (a) no fracture callus formation, (b) fibrotic scar tissue within the fracture, (c) diminished angiogenesis, and (d) poor mechanical performance, all of which are consistent with clinical manifestations of the atrophic nonunion patients. The results of these experiments demonstrated the utility of the mouse RA nonunion model with high clinical relevance. The mouse RA nonunion model provided a useful tool to study the pathology of atrophic nonunion under inflammation.

### Example 2: Inflammation Reduces Expression of Angiogenic Factors and Impairs Angiogenesis in Mice

To study the cellular and molecular basis of the negative impact of inflammation on angiogenesis in bone fracture healing, the following experiments were conducted using the mouse RA nonunion model described above.

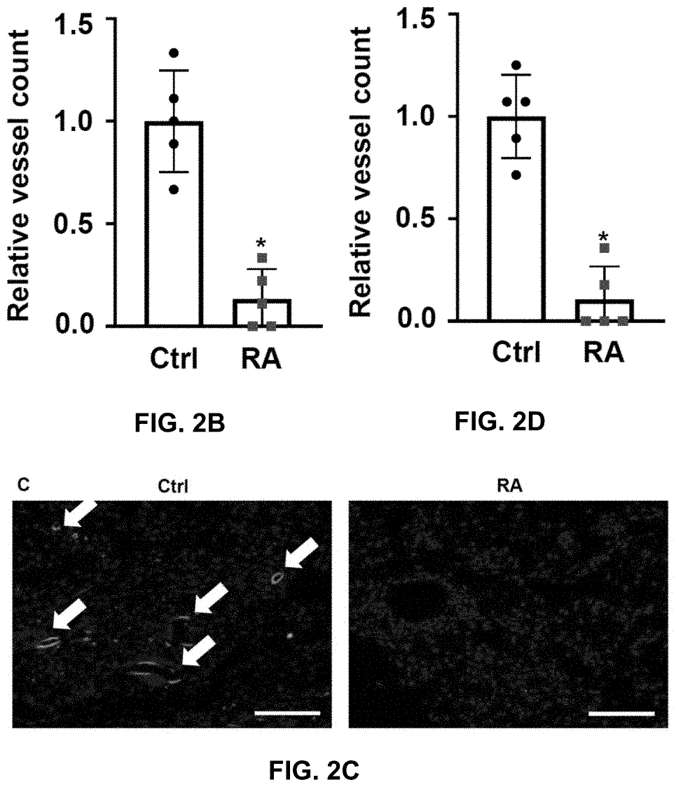

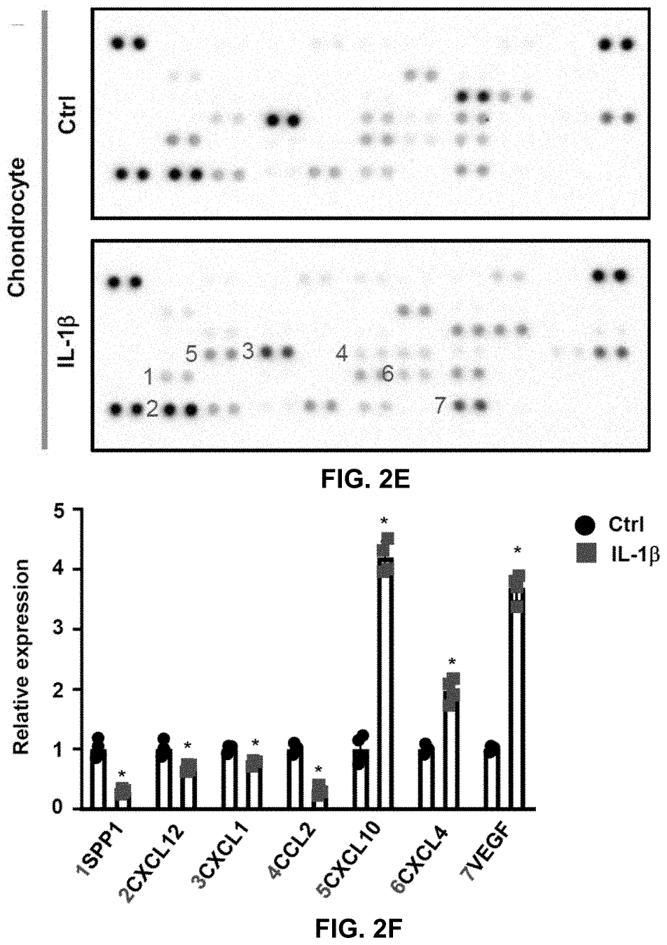

Clinical studies and rodent models have established that the defect of vascularization coincides with fracture nonunion, especially under inflammatory conditions, such as RA. Consistent with these reports, we observed that the control mice formed blood vessels at 10 dpf, the peak angiogenic time point in murine fracture healing. In contrast, the RA mice had diminished angiogenesis and poor angiogenic connectivity in 10dpf callus tissues (FIG. 2A). Quantification confirmed significantly fewer blood vessels in the RA fracture callus at 10 dpf (FIG. 2B).

We also performed immunohistochemistry (IHC) to detect Endomucin positive blood vessels in fracture callus. Similar to the angiography results, IHC revealed almost nondetectable blood vessels in the RA fracture callus, but abundant blood vessels were observed in 10 dpf control fracture callus (FIGS. 2C and 2D). These data indicate that systemic inflammation has adverse effects on the expression of angiogenic factors, leading to reduced angiogenesis during fracture repair. Since chondrocytes and osteoblasts are the primary cells that secrete angiogenic factors in fracture callus to stimulate blood vessel formation, we isolated primary chondrocytes and osteoblasts and treated them with IL-1β in vitro for 72 hours to examine the effect of inflammation on the production of angiogeic factors by these cells. A protein array (53 angiogenic factors) was used to determine the presence of angiogenic mediators in the culture supernatant from chondrocytes and osteoblasts, respectively. Inflammation significantly reduced the expression of angiogenic factors in chondrocytes, including secreted phosphoprotein 1 (SPP1), C-X-C motif chemokine ligand 12 (CXCL12), C-X-C motif chemokine ligand 1 (CXCL1), and C-C motif chemokine ligand 2 (CCL2), but induced the expression of C-X-C motif chemokine ligand 4 (CXCL4), C-X-C motif chemokine ligand 10 (CXCL10) and vascular endothelial growth factor (VEGF) (FIGS. 2E and 2F).

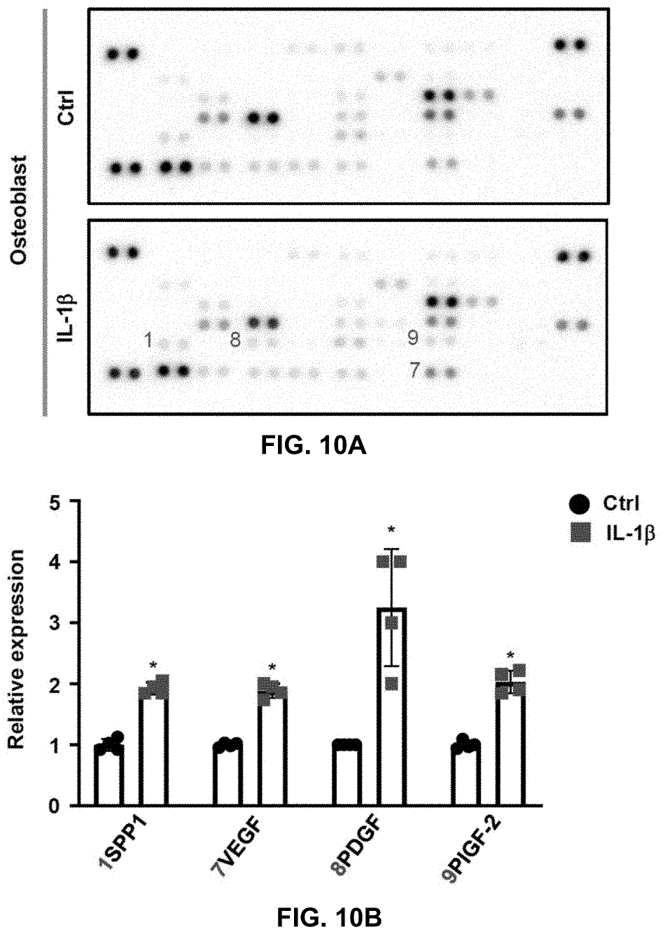

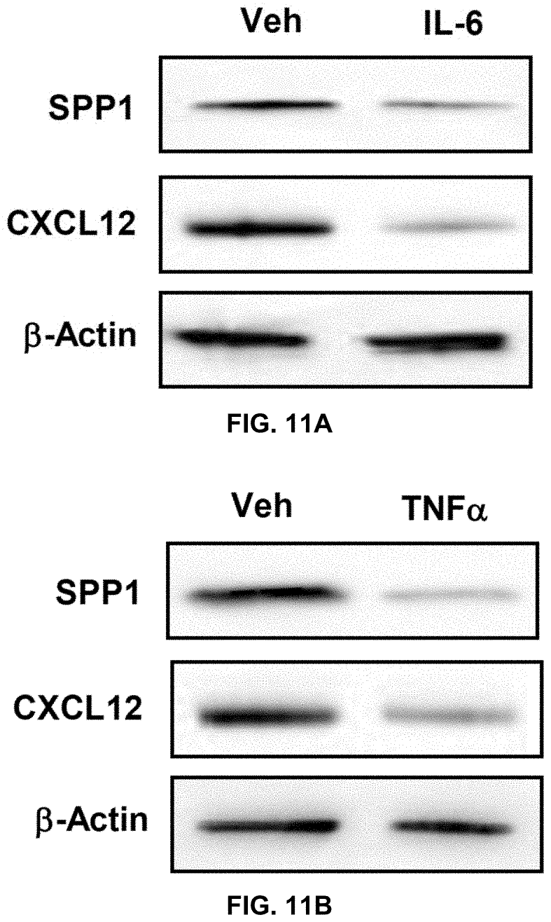

Surprisingly, expression of angiogenic factors from osteoblasts was minimally altered by IL-1β treatment (FIG. 10A). The expression of SPP1, VEGF, platelet-derived growth factor (PDGF) and placental growth factor 2 (PIGF-2) was even significantly increased under IL-1β treatment in primary osteoblasts (FIG. 10B). These data highlight the key role of chondrocytes in angiogenesis defect observed in RA mice during fracture nonunion. Since other inflammatory cytokines, such as IL-6 and TNFα have been shown up-regulated in RA fracture callus (FIG. 8), we also treated primary chondrocytes with IL-6 and TNFα. Similar to the effect of IL-1β, treatments of IL-6 and TNFα could also reduce the protein expression of SPP1 and CXCL12 in chondrocytes (FIG. 11).

Although the failure of bone healing is due to the interplay of multiple components, including defects of growth factors, progenitor cells, and mechanical factors, lack of blood supply has been long believed as an essential trigger to fracture healing defect, particularly atrophic nonunion. Clinical studies over the past decade revealed a three-fold increase in the nonunion rate in patients with ischemic injuries to tibia fracture compared to the average fracture patients. More importantly, fracture nonunion can be significantly improved by reconstruction of the vascular structure and restoration of blood supply, suggesting the pivotal role of angiogenesis on the development of fracture nonunion.

Recent studies have implicated that chondrocytes and osteoblasts are two major cell sources secreting angiogenic factors to restore the blood supply via angiogenesis during the initial fracture repair process. Restored blood supply then brings growth factors and oxygen to facilitate bony callus formation through chondrogenic and osteogenic differentiation of progenitor cells, and meanwhile brings osteoclasts to remodel the callus to regain the normal bone structure. Despite these findings, the underlying mechanism by which impaired angiogenesis results in atrophic nonunion remains largely unknown, especially under pathological inflammation conditions.

We comprehensively screened the angiogenic factors secreted from chondrocytes and osteoblasts under IL-1β treatment. Surprisingly, unlike chondrocytes, IL-1β induced the expression of the angiogenic factors in osteoblasts. In addition, IL-1β significantly induced VEGF expression in both chondrocytes and osteoblasts. Several studies have demonstrated that VEGF and VEGF signaling are the potent angiogenic factor and pathway to stimulate vessel formation and fracture repair in mice. However, our in vivo finding is that the RA mice developed impaired angiogenesis due to inflammation, which leads us to speculate that the reduced angiogenic factors, but not VEGF, likely are the direct downstream targets mediating the reduced angiogenesis observed in vivo. More importantly, SPP1 and CXCL12 were identified as two angiogenic factors most reduced in IL-1β treated chondrocytes. Their physiologic importance was evident in HUVEC experiments that showed that the addition of SPP1 and CXCL12 restored angiogenic defect present in supernatants harvested from IL-1β treated chondrocyte cultures. These findings indicated that chondrocytes are the target cells that mediate the angiogenic defect observed in the RA mice and that SPP1 and CXCL12 are potential downstream targets of inflammation in chondrocytes.

The results of these experiments uncovered several key findings that highlight a translational implication to treat atrophic nonunion. Through the screening of angiogenic factors, in vitro angiogenesis assays, and the RA fracture nonunion model, we demonstrated that inflammation reduced the expression of SPP1 and CXCL12 in chondrocytes and lead to diminished angiogenesis and atrophic nonunion in the RA mouse model.

### Example 3: Spp1 and Cxcl12 Restores Angiogenesis Under Inflammation in Vitro

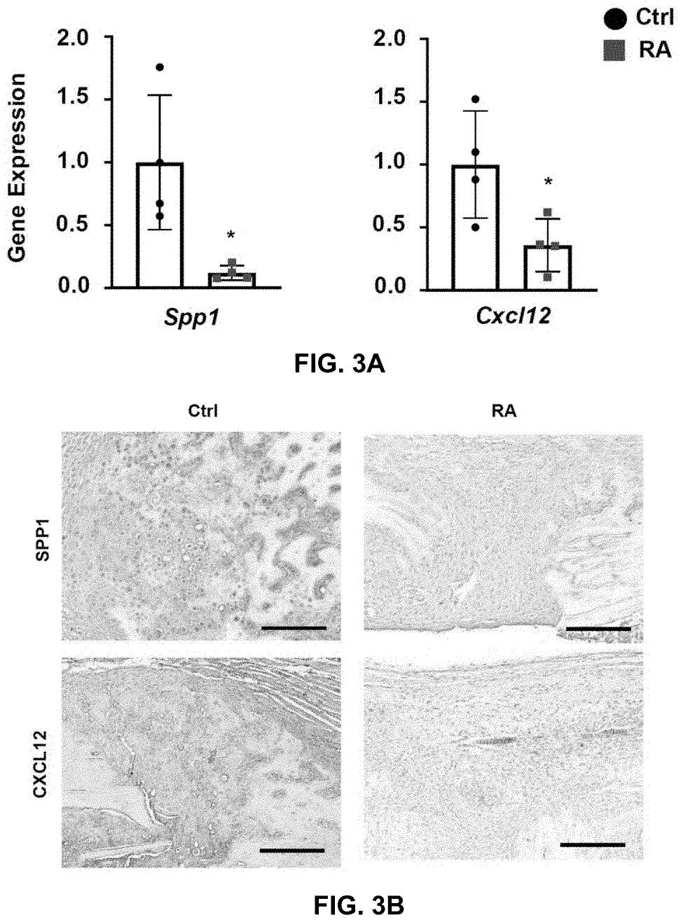

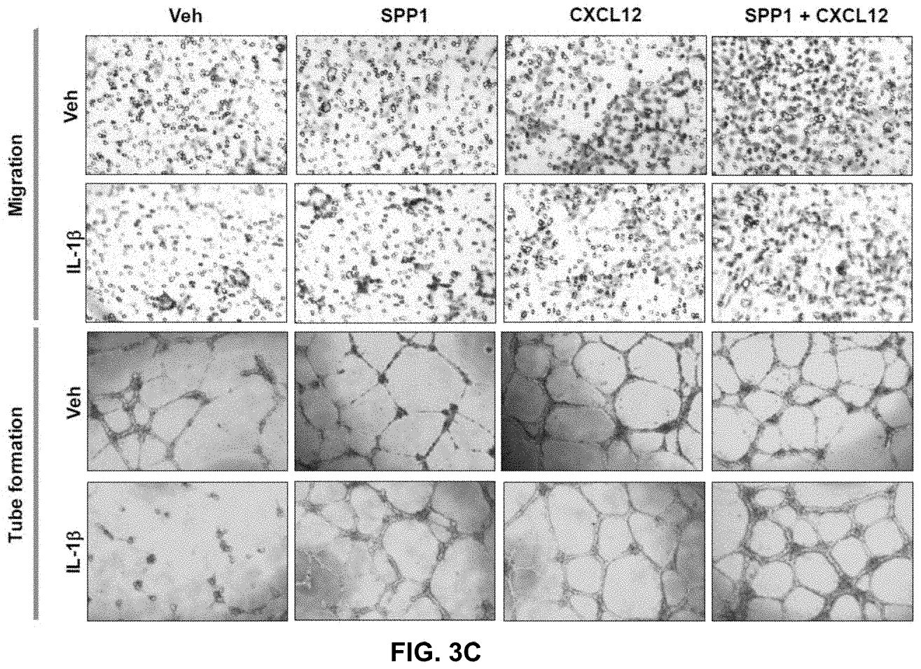

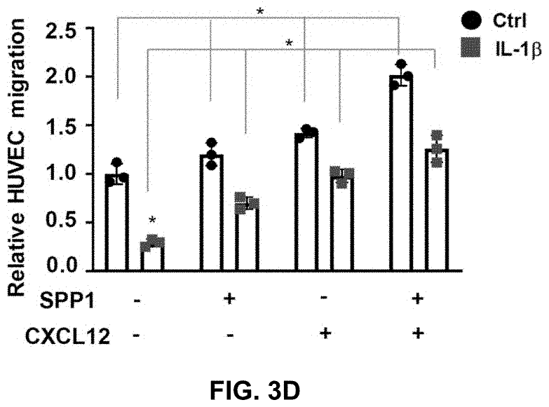

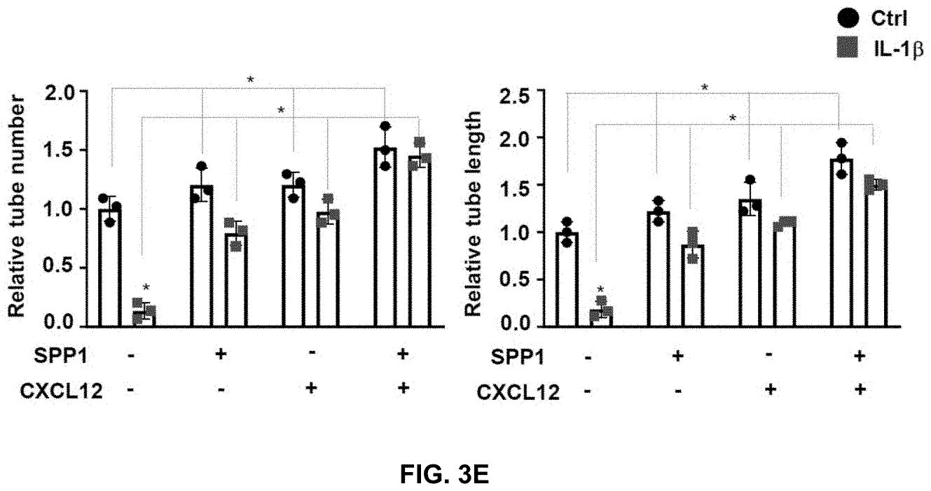

In Example 2 above, SPP1 was demonstrated to be the most reduced factor by IL-1β and CXCL12, which was the most abundantly expressed factor in chondrocytes, was also demonstrated to be significantly reduced, we focused on these two angiogenic factors to examine whether SPP1 and CXCL12 were the downstream targets of inflammation that mediate the angiogenic defect. As described below, we confirmed that Spp1 and Cxcl12 gene expression was decreased in 10 dpf RA fracture callus compared to the control callus (FIG. 3A), in agreement with the decreased protein expression. IHC performed on 10 dpf fractured tissue revealed abundant expression of SPP1 and CXCL12 in the control fracture callus, yet an almost non-detectable expression of SPP1 and CXCL12 in the RA fracture callus (FIG. 3B), confirming the down-regulation of SPP1 and CXCL12 by inflammation in the context of fracture repair under RA conditions.

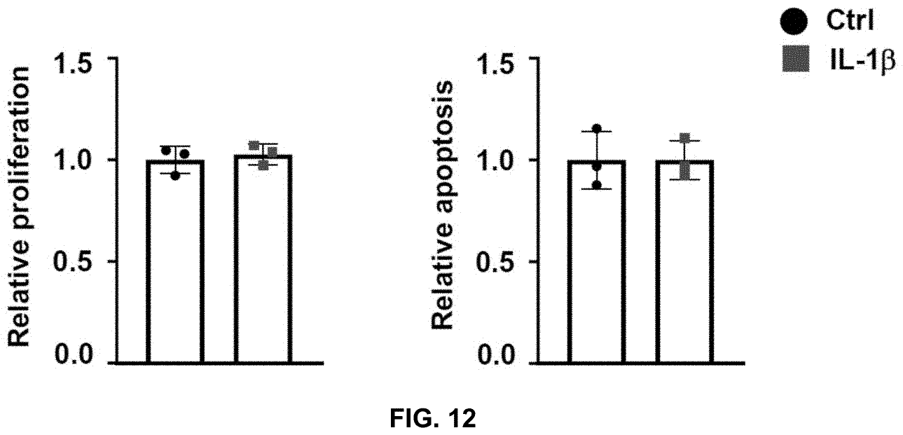

To confirm a physiological effect of inflammation on chondrocyte regulation of angiogenesis, we examined in vitro angiogenesis of human umbilical vein endothelial cells (HUVEC) using culture medium supernatants collected from primary chondrocytes treated with vehicle (control) or IL-1β (FIGS. 3C, 3D, and 3E). Consistent with our in vivo angiogenesis findings, the control culture supernatants from vehicle-treated chondrocytes had robust cell migration as well as an abundance of well-developed vessel tubes in vitro. In contrast, a significant reduction of HUVEC migration, tube number, and tube length was observed in the presence of culture supernatant from IL-1β-treated chondrocytes. This observation was not related to the continued presence of IL-1β in the supernatant since the angiogenic factors protein array results confirmed that there was no IL-1β left in the culture medium after 72 hours of IL-1β treatment (FIG. 2E). Additionally, compared to culture medium from vehicle-treated chondrocytes, culture medium from IL-1β-treated chondrocytes did not induce any apparent difference in HUVEC cell proliferation and apoptosis (FIG. 12), suggesting decreased angiogenic capacity of the culture medium was at least partially attributed to reduced SPP1 and CXCL12, but not dysfunction of HUVEC itself.

We applied 500 ng/ml of SPP1 and 100 ng/ml of CXCL12 to the chondrocyte culture medium and performed HUVEC angiogenesis assays. As expected, administration of SPP1 and CXCL12 individually, or in combination, to culture medium from vehicle-treated chondrocytes resulted in induction of HUVEC migration and tube formation. More importantly, exogenous SPP1 and CXCL12 administration restored angiogenic capacity, as reflected by increased cell migration, tube number and tube length under culture medium from IL-1β treated chondrocytes. We also observed that combined administration of SPP1 and CXCL12 achieved the most profound effect on the restoration of HUVEC in vitro angiogenesis. These data, together with the murine RA fracture nonunion model results described above, identified the reduction of SPP1 and CXCL12 as the molecular basis of the negative impact of inflammation on angiogenesis.

The results of these experiments demonstrated that the administration of exogenous SPP1 and CXCL12 represents a potential therapeutic approach for the treatment of fracture nonunion under inflammatory conditions. The physiologic importance of SPP1 and CXCL12 was evident in HUVEC experiments that showed that the addition of SPP1 and CXCL12 ameliorated the angiogenic defect present in supernatants harvested from IL-1β treated chondrocyte cultures. These findings indicated that chondrocytes were the target cells that mediated the angiogenic defect observed in the RA mice and that SPP1 and CXCL12 are potential downstream targets of inflammation in chondrocytes.

The results of these experiments demonstrated that SPP1 and CXCL12 were downstream targets of inflammation and that supplementation of SPP1 and CXCL12 restored angiogenic capacity in vitro.

### Example 4: Delivery of Spp1 and Cxcl12 Via Biodegradable Scaffold

In order to promote angiogenesis at the fracture sites while avoiding excessive angiogenesis in other tissues, a sustained release formulation to deliver SPP1 and CXCL12 locally was developed as described below. The sustained-release formulation made use of a polycaprolactone (PCL) scaffold. PCL is a US Food and Drug Administration (FDA) approved biodegradable polymer for tissue engineering applications.

An electrospinning and electrospraying system was used to fabricate PCL scaffolds by simultaneously electrospinning PCL fibers and electrospraying poly(lactide-co-glycolide) (PLGA) microspheres loaded with SPP1 (100 μg/ml) and/or CXCL12 (20 μg/ml) (FIG. 4A).

To characterize the core-shell structured microspheres, we added Rhodamine B marker to the PLGA solution and added Hoechst marker to the growth factor solution, and obtained confocal images as described above. The fluorescent images shown in FIG. 4B confirmed that the microspheres assumed a core-shell structure with PLGA as the shell and SPP1 or CXCL12 as the core (FIG. 4B).

The scaffold structure and microsphere distribution were characterized by scanning electron microscope (SEM). The PCL scaffold was formed by interlaced fibers and the microspheres with a diameter of about 5 μm were distributed uniformly in the scaffolds (FIG. 4C). Mechanical testing of the scaffold was conducted as described above. The scaffolds exhibited a tensile strength of 28.8 MPa and Young's modulus of 111.5 MPa (FIG. 13).

The release profiles of SPP1 and CXCL12 in vitro were monitored over a 4-week period as described above. According to the ELISA assays of the collected medium, SPP1 and CXCL12 exhibited a two-stage release pattern (FIG. 4D). Both compounds displayed burst release in the first three days followed by a controlled slow release until day 28. The release of SPP1 and CXCL12 from the scaffold loaded with both SPP1 and CXCL12 was slower than the corresponding release profiles from the scaffolds loaded with SPP1 only or CXCL12 only (FIG. 4D).

### Example 5: Release of Spp1 and Cxcl12 Promotes Angiogenesis Under Inflammatory Conditions

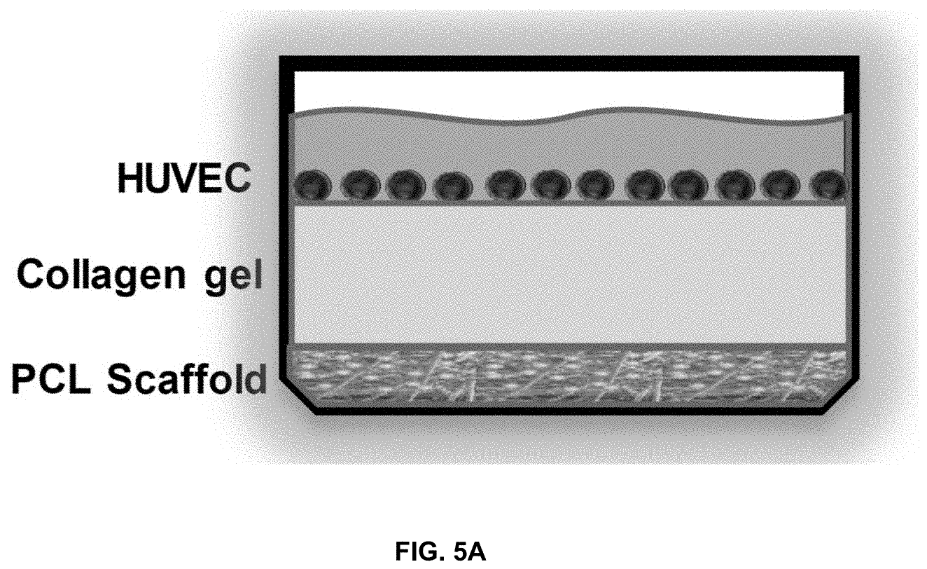

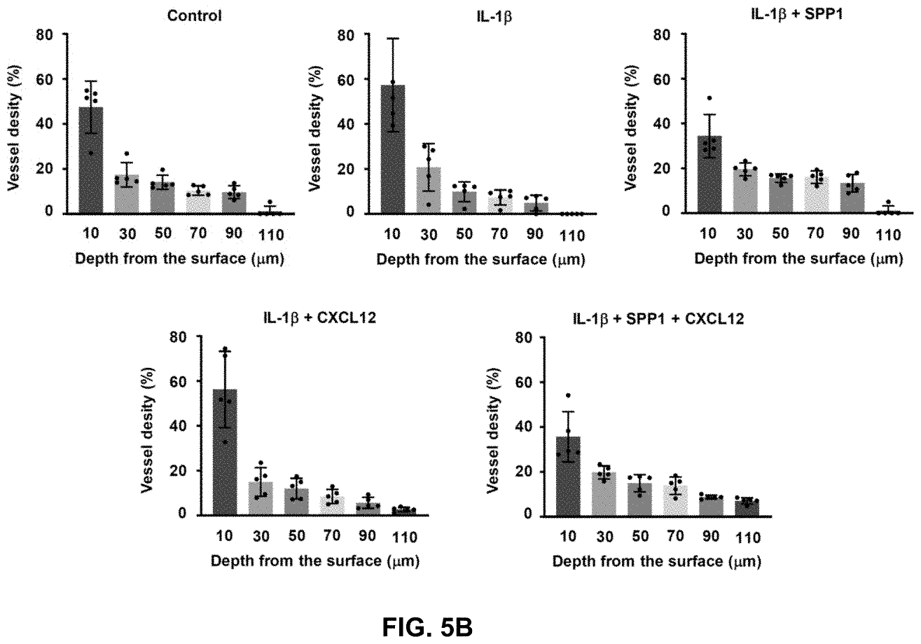

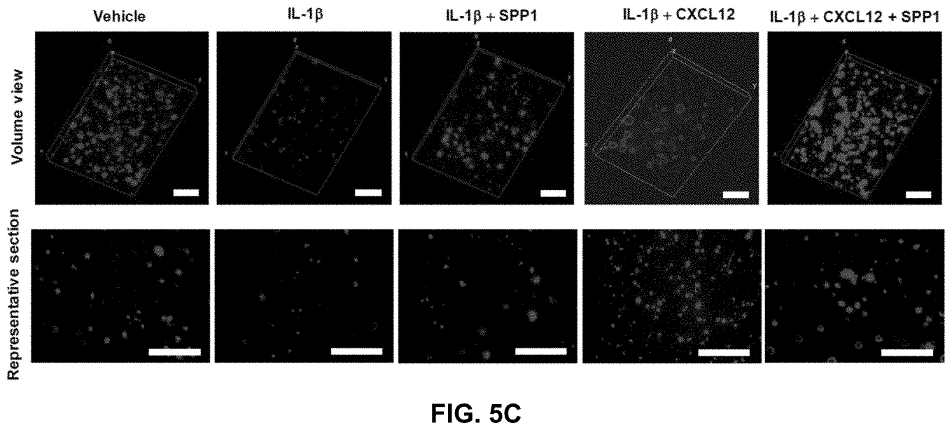

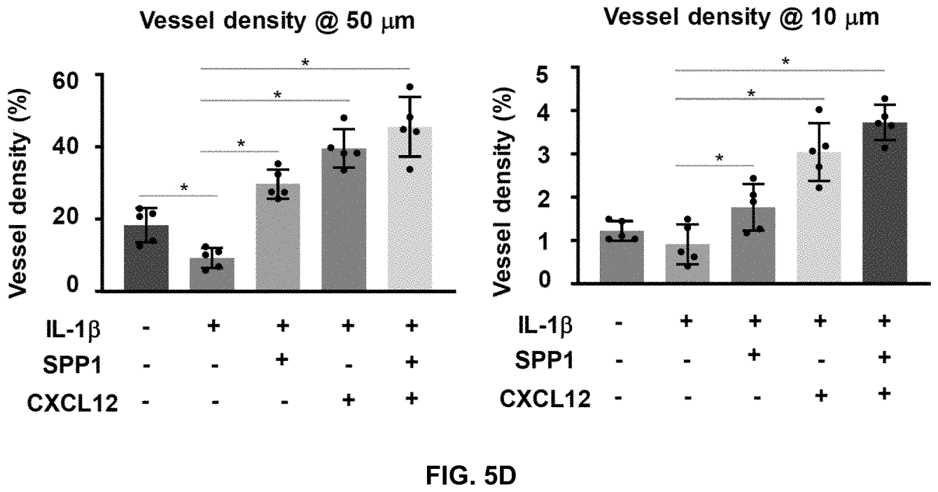

Since the RA mice exhibited a systemic inflammatory environment, particularly the increased expression of inflammatory cytokines was identified in the fracture sites (FIG. 8), the following experiments were conducted to examine whether SPP1 and CXCL12 released from the scaffolds described above promoted HUVEC angiogenic capacity under the inflammatory conditions. We mimicked the scenario when the scaffold was implanted at the fracture site by placing the collagen gel on the top of PCL scaffolds loaded with SPP1 and/or CXCL12 and then culturing CM-Dil dye-labeled HUVEC on the collagen gel under the control or inflammatory conditions (FIG. 5A). The migration of HUVEC cells on the collagen gel was then examined by confocal microscopy. As expected, IL-1β treatment significantly reduced the HUVEC migration, particularly in the deep zone from 50 μm to 110 μm from the top surface (FIG. 5B). However, growth factors substantially promoted the migration under IL-1β treatment (FIG. 5B). The SPP1 released from the scaffold loaded with SPP1 alone more greatly promoted HUVEC migration than the CXCL12 released from the scaffold loaded with CXCL12 alone. The greatest cell migration was found in the group with combined SPP1 and CXCL12 release, especially in the deep zone up to 110 μm from the top surface (FIG. 5B).

In addition to cell migration, we also examined the tube formation based on the lumens formed in the collagen gel, particularly focused on the area with a majority of HUVEC cells. IL-1β treatment significantly reduced the tube formation as reflected by decreased lumen formation in the collagen gel, in the top surface (10 μm) and deep zone (50 μm), although no HUVEC migration difference was observed in the surface under IL-1β treatment. Importantly, the lumen formation was restored in the groups where scaffolds were loaded with SPP1 and CXCL12, even under inflammatory conditions. The lumen density in the group with only CXCL12 release was significantly higher than the group with only SPP1 release. The group with both SPP1 and CXCL12 had significantly greater lumen density than the group with CXCL12 release alone in both surface area and deep zone (FIGS. 5C and 5D).

The results of these experiments confirmed that SPP1 and CXCL12 were gradually released for 4 weeks from the scaffolds, which would potentially benefit angiogenesis and inflammatory bone fracture healing.

### Example 6: Controlled Release of Spp1 and Cxcl12 Restores Angiogenesis and Fracture Union in the RA Mice

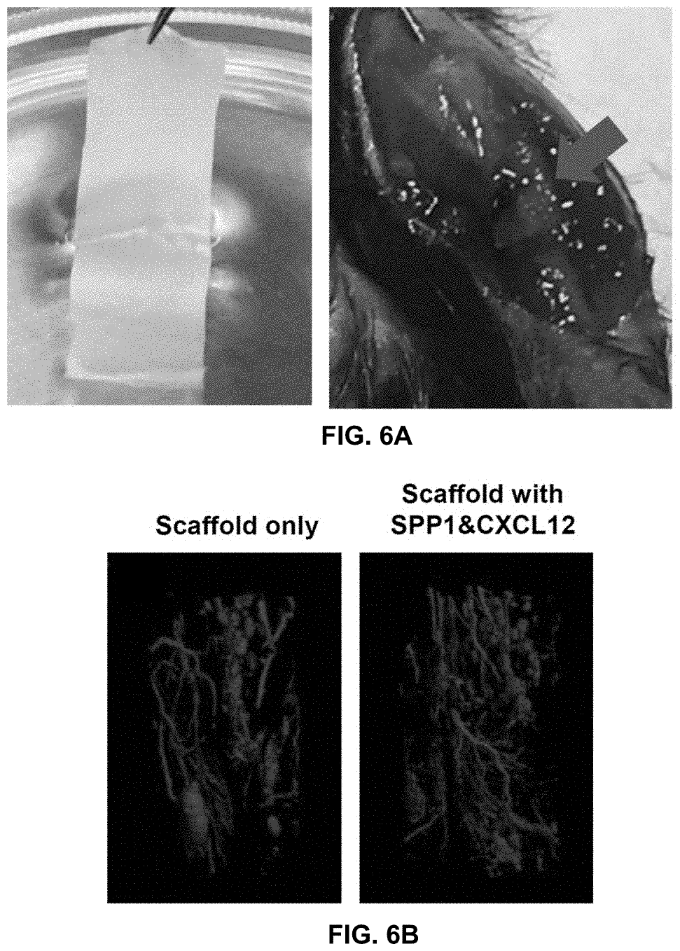

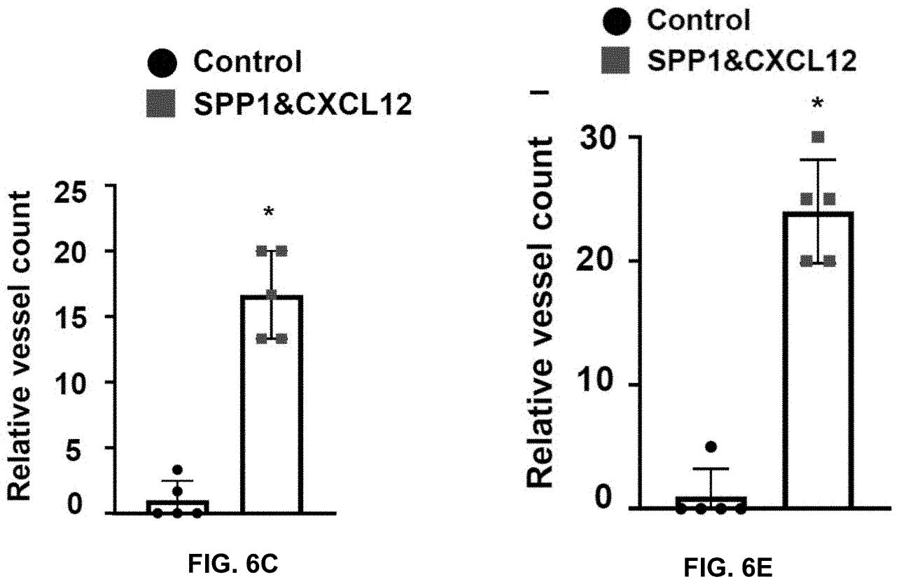

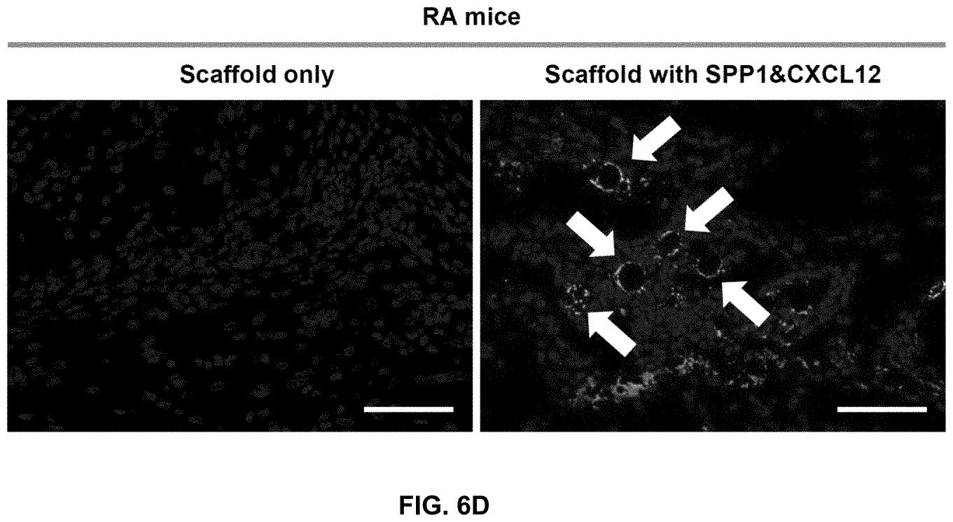

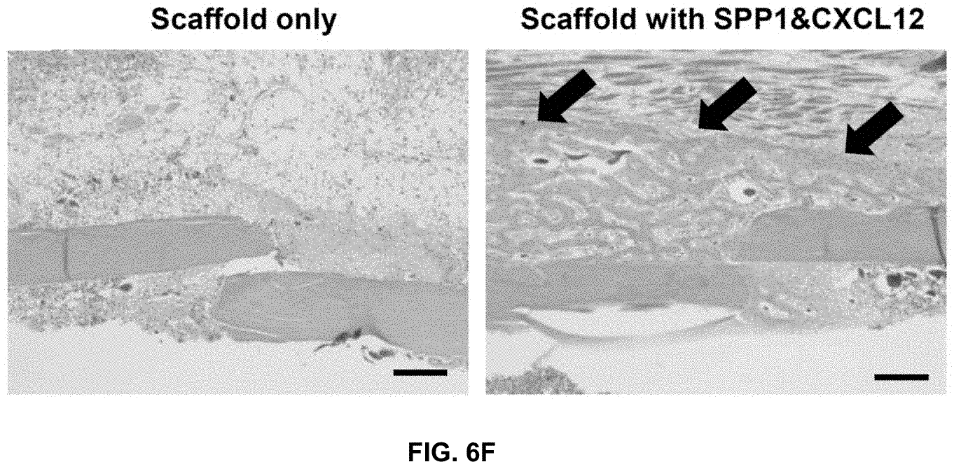

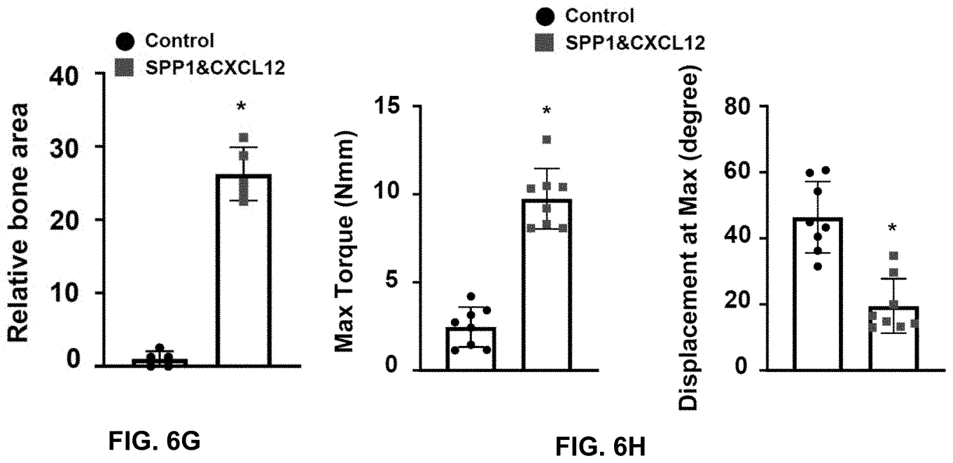

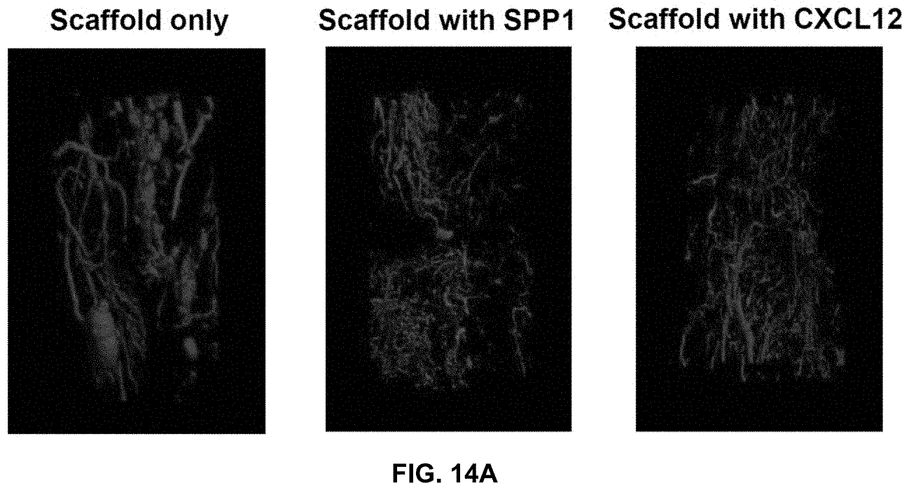

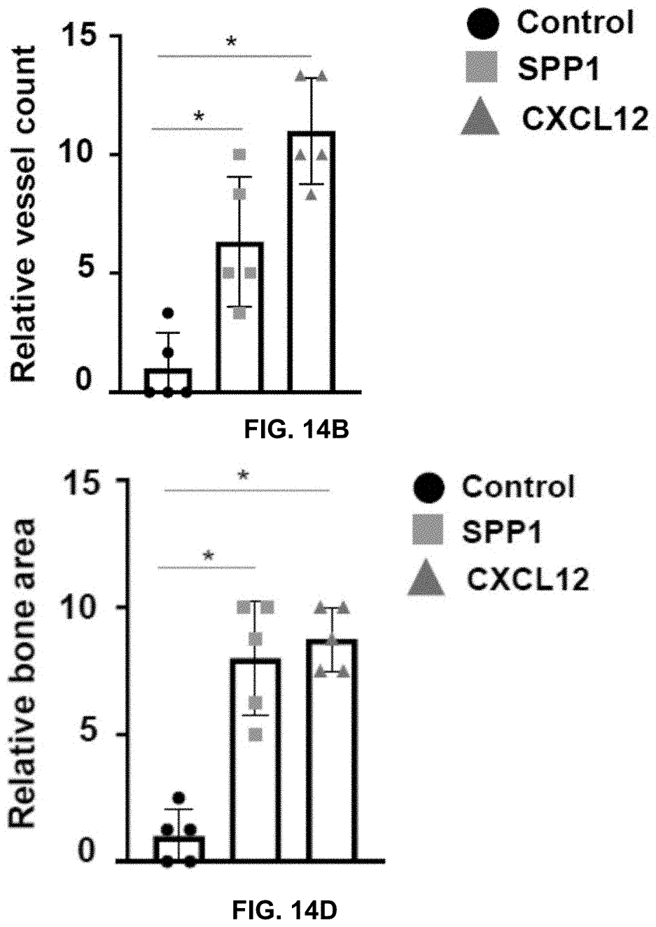

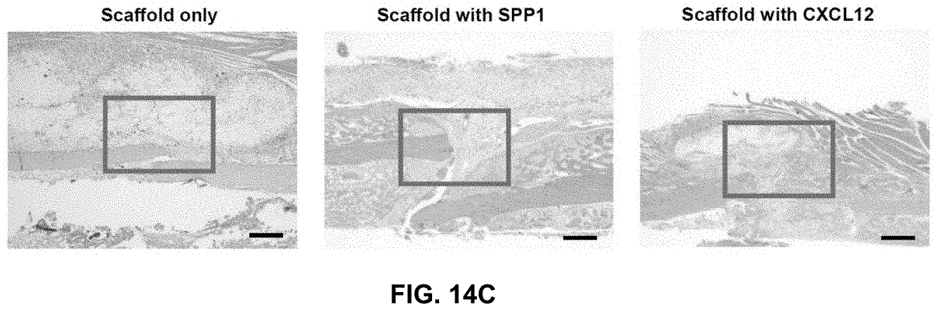

To characterize the effects of treatment using the scaffold composition described above in vivo, the following experiments were conducted. We applied the scaffolds with SPP1 (100 μg/ml) and/or CXCL12 (20 μg/ml) to treat fracture nonunion in the RA mice described above. A 2-mm thickness of the scaffold described above was wrapped around the fractured bone of the RA mice immediately after the fracture procedure (FIG. 6A). Vascular structure and fracture healing process were evaluated 10 days after application of scaffolds by microCT and histology, respectively. Similar to the RA mice, diminished angiogenesis was observed at 10 dpf callus tissue applied with PCL scaffold without growth factors under the systemic inflammation in vivo. In contrast, PCL scaffold loaded with SPP1 and/or CXCL12 significantly induced angiogenic response in 10 dpf callus and resulted in the formation of more blood vessels under the RA condition (FIGS. 14A, 14B, 6B, and 6C). Consistent with the observation from in vitro angiogenesis assays, SPP1 and CXCL12 in combination achieved the most effect on the restoration of blood vessel formation in the RA mice, compared to the individual SPP1 and CXCL12 treatment.

We also performed immunohistochemistry (IHC) to detect Endomucin positive blood vessels in 10 dpf fracture callus and revealed significantly more blood vessels in the RA fracture callus applied with PCL scaffold containing both SPP1 and CXCL12 (FIGS. 6D and 6E). More importantly, coincident with the restoration of blood vessels in fracture callus, newly formed woven bone was also observed in the RA mice treated with SPP1 and CXCL12 via PCL scaffold. Individual SPP1 and CXCL12 treatment similarly induced more woven bone (8 folds increase) in the adjacent region of fracture at 10 dpf (FIGS. 14C and 14D). Application of SPP1 and CXCL12 together increased woven bone in 10 dpf callus by 25 folds. Notably, the newly formed bone replaced the fibrotic tissue and unified the fracture nonunion in the RA mice after 10 days of local treatment with SPP1 and CXCL12 (FIGS. 6F and 6G). Finally, we measured the bone biomechanical properties by the torsion test and found that the maximum bone strength was significantly restored by 28 dpf in the RA mice treated with SPP1 and CXCL12 (FIG. 6H). Hence, these findings strongly suggest that local release with SPP1 and CXCL12 via PCL scaffold presents an effective therapeutic approach to treat impaired angiogenesis and fracture nonunion under inflammatory conditions.

In these experiments, the biodegradable scaffold loaded with SPP1 and CXCL12 displayed a beneficial effect on angiogenesis and fracture repair in mice despite the presence of inflammation. These findings strongly suggested that sustained release of SPP1 and CXCL12 represents an effective therapeutic approach to treat impaired angiogenesis and fracture nonunion under inflammatory conditions.

Finally, as proof of concept that administration of SPP1 and CXCL12 can restore angiogenesis and be beneficial for atrophic nonunion fracture healing, we applied the PCL scaffold with SPP1 and CXCL12 to the fracture site in the RA mice. It is well established that excessive SPP1 and CXCL12 are associated with cancer angiogenesis, metastasis, and malignancy. In addition, excessive angiogenesis mediated by SPP1 and CXCL12 in the synovium may exacerbate the joint destruction in RA patients. Therefore, in order to avoid potential side effects, we engineered a PCL scaffold for the sustained release of SPP1 and CXCL12 locally in the fracture site. PCL scaffold is an FDA approved biodegradable material and has been used in bone regeneration in animals. The fabricated scaffolds were fibrous, mimicking the morphology of the extracellular matrix in the periosteum tissue. Notably, the scaffolds had comparable Young's modulus and tensile strength to the periosteum tissue. As expected, treatment with SPP1 or CXCL12 alone showed beneficial effects on angiogenesis and new bone formation in RA callus. More importantly, consistent with in vitro findings, the combination of SPP1 and CXCL12 exhibited a synergistic effect in vivo, i.e., significantly induced angiogenesis greater than either factor alone. Surprisingly, both factors together also induced bony callus formation and fracture union at 10 days post-fracture in the RA mice. While individual delivery of SPP1 or CXCL12 treatment restored the fracture repair in the RA mice with cartilage callus and with areas of new bone formation at 10 dpf fracture, the combination treatment gave rise to a more mature bony callus without residual cartilage, suggesting the accelerated fracture healing. The synergistic effect is likely due to the restoration of endomucin positive blood vessels by SPP1 and CXCL12, given the evidence that endomucin positive type H vessel is the most important vessel facilitating fracture callus ossification and union. In addition, it has been shown that SPP1 can signal through multiple integrins to facilitate vessel formation through activation of PI3K and MAP kinase pathways in endothelial cells. Similarly, CXCL12 can also activate the MAP kinase pathway through β-arrestin. Therefore, it is reasonable to speculate that SPP1 and CXCL12 synergistically induce angiogenesis in vitro and in vivo under inflammatory conditions through activation of MAP kinase pathways in endothelial cells. Moreover, SPP1 is predominantly synthesized by osteoblasts, therefore it is possible that increased vessel formation and bone formation is achieved through an SPP1 mediated positive feedback loop. CXCL12 can also induce osteoblast differentiation and mineralization, therefore accelerating bony callus formation in mice.

What is claimed is:

1. A formulation for treating a fracture non-union in a patient in need, the formulation comprising a biodegradable scaffold, a therapeutically effective amount of CXCL12, and a therapeutically effective amount of SPPI, wherein:
a. the biodegradable scaffold is configured to be applied over a region of the fracture non-union, within a gap of the fracture non-union, and any combination thereof; and
b. the biodegradable scaffold is configured for extended release of the therapeutically effective amounts of CXCL12 and SPPI.

2. The formulation of claim 1, wherein the biodegradable scaffold comprises a plurality of biodegradable polymer fibers, wherein the biodegradable polymer fibers comprise a scaffold polymer is selected from PCL, PTO, and PLGA.

3. The formulation of claim 2, wherein the scaffold polymer comprises PCL.

4. The formulation of claim 3, wherein the biodegradable scaffold comprises a thickness of about 100 μm.

5. The formulation of claim 4, wherein the biodegradable scaffold comprises a width of about 2 mm.

6. The formulation of claim 5, wherein the biodegradable scaffold comprises a tensile strength of about 29 MPa and a Young's modulus of about 111.5 MPa.

7. The formulation of claim 6, further comprising a plurality of biodegradable microspheres encapsulating the therapeutically effective amounts of CXCL12 and SPPI.

8. The formulation of claim 7, wherein the plurality of biodegradable microspheres comprise a diameter of about 5 μm.

9. The formulation of claim 8, wherein the plurality of biodegradable microspheres comprise a microsphere polymer selected from PCL, PTO, and PLGA.

10. The formulation of claim 9, wherein the microsphere polymer comprises PLGA.

11. The formulation of claim 10, wherein the plurality of biodegradable microspheres are uniformly distributed throughout the biodegradable scaffold.

12. The formulation of claim 11, wherein each biodegradable microsphere encapsulates a mixture comprising CXCL12 and SPPI.

13. The formulation of claim 12, wherein the plurality of biodegradable microspheres comprises a first portion of microspheres encapsulating CXCL12 and a second portion of microspheres encapsulating SPPI.

14. The formulation of claim 12, wherein the biodegradable scaffold is configured for sustained release of the therapeutically effective amounts of CXCL12 and SPPI over a period of about 4 weeks.

15. A method for treating a fracture non-union in a patient in need, the method comprising:
a. providing a formulation comprising a biodegradable scaffold, a therapeutically effective amount of CXCL12, and a therapeutically effective amount of SPPI, the scaffold configured for extended release of the therapeutically effective amounts of CXCL12 and SPPI; and
b. applying the biodegradable scaffold over a region of the fracture non-union, within a gap of the fracture non-union, and any combination thereof.

16. The method of claim 15, wherein the biodegradable scaffold comprises a plurality of biodegradable polymer fibers, the biodegradable polymer fibers comprising a scaffold polymer, the scaffold polymer comprising PCL.

17. The method of claim 16, wherein the biodegradable scaffold comprises a thickness of about 100 μm and a width of about 2 mm.

18. The method of claim 17, wherein the formulation further comprises a plurality of biodegradable microspheres uniformly distributed throughout the biodegradable scaffold, the plurality of biodegradable microspheres comprising PLGA encapsulating the therapeutically effective amounts of CXCL12 and SPPI.

19. The method of claim 18, wherein the biodegradable scaffold is configured for sustained release of the therapeutically effective amounts of CXCL12 and SPPI over a period of about 4 weeks.

20. The method of claim 19, further comprising modulating the therapeutically effective amounts of CXCL12 and SPPI by varying a number of layers of the biodegradable scaffold applied over the region of the fracture non-union, within a gap of the fracture non-union, and any combination thereof.
